(19) United States
(12) Patent Application Publication
(10) Pub. No.: US 2015/0259295 A1
(43) Pub. Date: Sep. 17, 2015

(54) SUBSTITUTED PYRAZOLE COMPOUNDS AS LPAR ANTAGONISTS

(71) Applicant: Hoffmann-La Roche Inc., Nutley, NJ (US)

(72) Inventors: Stephen Deems Gabriel, Morristown, NJ (US); Matthew Michael Hamilton, Hackettstown, NJ (US); Yimin Qian, Plainsboro, NJ (US); Achyutharao Sidduri, Newark, NJ (US)

(21) Appl. No.: 14/402,128

(22) PCT Filed: Jun. 17, 2013
(86) PCT No.: PCT/EP2013/062458

§ 371 Date: Nov. 19, 2014

Related U.S. Application Data
(60) Provisional application No. 61/661,958, filed on Jun. 20, 2012.

(51) Int. Cl. C07D 231/40; C07D 405/12

(52) U.S. Cl. CPC C07D 231/40; C07D 405/12

(57) ABSTRACT

Provided herein are compounds of the formula (I): as well as pharmaceutically acceptable salts thereof, wherein the substituents are as those disclosed in the specification. These compounds, and the pharmaceutical compositions containing them, are useful for the treatment of inflammatory diseases and disorders such as, for example, pulmonary fibrosis

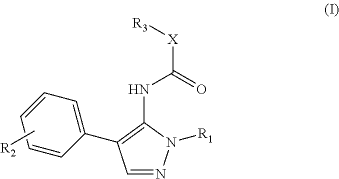

The present invention relates to organic compounds useful for therapy and/or prophylaxis in a mammal of an inflammatory disease or disorder, and in particular to substituted compounds, their manufacture, pharmaceutical compositions containing them and their use as lysophos-phatidic acid (LPA) antagonists.

LPA is a family of bioactive phosphate lipids which function like a growth factor mediator by interacting with LPA receptors, a family of G-protein-coupled receptors (GPCRs). The lipid family has long chain saturated (such as C18:0 or C16:0) or unsaturated (C18:1 or C20:4) carbon chains attached to the glycerol through an ester linkage. In biological systems, LPA is produced by multi-step enzymatic pathways through the de-esterification of membrane phospholipids. Enzymes that contribute to LPA synthesis include lysophospholipase D (lysoPLD), autotaxin (ATX), phospholipase A1 (PLAT), phospholipase A2 (PLA2) and acylglycerol kinase (AGK) (British J. of Pharmacology 2012, 165, 829-844).

There are at least six LPA receptors identified (LPAR1-6). LPA signaling exerts a broad range of biological responses on many different cell types, which can lead to cell growth, cell prolifera-tion, cell migration and cell contraction. Up regulation of the LPA pathway has been linked to multiple diseases, including cancer, allergic airway inflammation, and fibrosis of the kidney, lung and liver. Therefore, targeting LPA receptors or LPA metabolic enzymes could provide new approaches towards the treatment of medically important diseases that include neuropsychiatric disorders, neuropathic pain, infertility, cardiovascular disease, inflammation, fibrosis, and cancer (Annu Rev. Pharmacol. Toxicol. 2010, 50, 157-186; J. Biochem. 2011, 150, 223-232).

Fibrosis is the result of an uncontrolled tissue healing process leading to excessive accumulation of extracellular matrix (ECM). Recently it was reported that the LPA1 receptor was over expressed in idiopathic pulmonary fibrosis (IPF) patients. Mice with LPA1 receptor knockout were protected from bleomycin-induced lung fibrosis (Nature Medicine 2008, 14, 45-54). Thus, antagonizing LPA1 receptor may be useful for the treatment of fibrosis, such as renal fibrosis, pulmonary fibrosis, arterial fibrosis and systemic sclerosis.

In an embodiment of the present invention, provided are compounds of general formula (I):

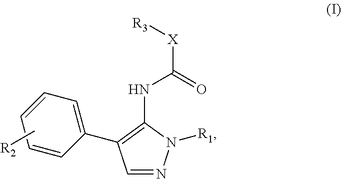

wherein:
X is oxygen, nitrogen or carbon;
R₁ is lower alkyl;
R₂ is hydrogen, halogen, —CH₂C(O)OH, alkoxy, cycloalkylcarboxylic acid, unsubstituted phenyl or phenyl substituted with halogen, —CH₂C(O)OH, cyclopropanecarboxylic acid, cyclopropanecarboxylic acid ethyl ester, methanesulfonylaminocarbonyl or tetrazole; and R₃ is cyclobutyl, oxetanyl, unsubstituted lower alkyl, lower alkyl substituted with unsubstituted phenyl or lower alkyl substituted with phenyl substituted with halogen or —CF₃, or a pharmaceutically acceptable salt thereof.

In a further embodiment of the invention, provided is a pharmaceutical composition comprising a therapeutically effective amount of a compound according to formula (I) and a therapeutically inert carrier.

In a still further embodiment of the invention, provided is a method for the treatment or prophylaxis of pulmonary fibrosis, which method comprises the step of administering a therapeutically effective amount of a compound according to formula (I) to a patient in need thereof.

All documents cited to or relied upon below are expressly incorporated herein by reference.

Unless otherwise indicated, the following specific terms and phrases used in the description and claims are defined as follows:

As used herein, the term “alkyl”, alone or in combination with other groups, refers to a branched or straight-chain monovalent saturated aliphatic hydrocarbon radical of one to twenty carbon atoms, preferably one to sixteen carbon atoms, more preferably one to ten carbon atoms.

The term “lower alkyl”, alone or in combination with other groups, refers to a branched or straight-chain alkyl radical of one to nine carbon atoms, preferably one to six carbon atoms, more preferably one to four carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like.

The term “cycloalkyl” refers to a monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, norbornyl, adamantyl and the like. In a preferred embodiment, the “cycloalkyl” moieties can optionally be substituted with one, two, three or four substituents, with the understanding that said substituents are not, in turn, substituted further. Each substituent can independently be, alkyl, alkoxy, halogen, amino, hydroxyl or oxygen (O═) unless otherwise specifically indicated. Examples of cycloalkyl moieties include, but are not limited to, optionally substituted cyclopropyl, optionally substituted cyclobutyl, optionally substituted cyclopentyl, optionally substituted cyclopentenyl, optionally substituted cyclohexyl, optionally substituted cyclohexylene, optionally substituted cycloheptyl, and the like or those which are specifically exemplified herein.

The term “heterocycloalkyl” denotes a mono- or polycyclic alkyl ring, wherein one, two or three of the carbon ring atoms is replaced by a heteroatom such as N, O or S. Examples of heterocycloalkyl groups include, but are not limited to, morpholinyl, thiomorpholinyl, piperazinyl, piperidinyl, pyrrolidinyl, tetrahydropyranyl, tetrahydrofuranyl, 1,3-dioxanyl and the like. The heterocycloalkyl groups may be unsubstituted or substituted and attachment may be through their carbon frame or through their heteroatom(s) where appropriate, with the understanding that said substituents are not, in turn, substituted further.

The term “aryl” refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthalene, 1,2-dihydronaphthalene, indanyl, 1H-indenyl and the like.

The term “heteroaryl,” refers to an aromatic mono- or polycyclic radical of 5 to 12 atoms having at least one aromatic ring containing one, two, or three ring heteroatoms selected from N, 0, and S, with the remaining ring atoms being C. Examples of such groups include, but are not limited to, pyridine, thiazole and pyranyl.

The alkyl, lower alkyl, aryl and heteroaryl groups described above may be substituted independently with one, two, or three substituents, with the understanding that said substituents are not, in turn, substituted further. Substituents may include, for example, halogen, lower alkyl, —CF₃, —SO₂CH₃, alkoxy, —C(O)CH₃, —OH, —SCH₃ and —CH₂CH₂OH.

As used herein, the term “alkoxy” means alkyl-O—; and “alkoyl” means alkyl-CO—. Alkoxy substituent groups or alkoxy-containing substituent groups may be substituted by, for example, one or more alkyl groups, with the understanding that said substituents are not, in turn, substituted further.

As used herein, the term “halogen” means a fluorine, chlorine, bromine or iodine radical, preferably a fluorine, chlorine or bromine radical, and more preferably a fluorine or chlorine radical.

Compounds of formula I can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbents or eluant). The invention embraces all of these forms.

As used herein, the term “pharmaceutically acceptable salt” means any pharmaceutically acceptable salt of the compound of formula (I). Salts may be prepared from pharmaceutically acceptable non-toxic acids and bases including inorganic and organic acids and bases. Such acids include, for example, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. Particularly preferred are fumaric, hydrochloric, hydrobromic, phosphoric, succinic, sulfuric and methanesulfonic acids. Acceptable base salts include alkali metal (e.g. sodium, potassium), alkaline earth metal (e.g. calcium, magnesium) and aluminum salts.

In the practice of the method of the present invention, an effective amount of any one of the compounds of this invention or a combination of any of the compounds of this invention or a pharmaceutically acceptable salt thereof, is administered via any of the usual and acceptable methods known in the art, either singly or in combination. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form or solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

Useful pharmaceutical carriers for the preparation of the compositions hereof, can be solids, liquids or gases. Thus, the compositions can take the form of tablets, pills, capsules, supposetories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingre-dient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

The dose of a compound of the present invention depends on a number of factors, such as, for example, the manner of administration, the age and the body weight of the subject, and the condition of the subject to be treated, and ultimately will be decided by the attending physician or veterinarian. Such an amount of the active compound as determined by the attending physician or veterinarian is referred to herein, and in the claims, as a “therapeutically effective amount”. For example, the dose of a compound of the present invention is typically in the range of about 1 to about 1000 mg per day. Preferably, the therapeutically effective amount is in an amount of from about 1 mg to about 500 mg per day.

In one embodiment the present invention provides a compound according to formula (I), wherein X is oxygen.

In another embodiment, provided is a compound according to formula (I), wherein R₁ is methyl.

In another embodiment, provided is a compound according to formula (I), wherein R₂ is hydrogen, —F, —Cl, —CH₂C(O)OH, methoxy, ethoxy, cyclopropanecarboxylic acid, unsubstituted phenyl, or cyclohexaneacetic acid

In another embodiment, provided is a compound according to formula (I), wherein R₂ is phenyl substituted with —CH₂C(O)OH, cyclopropanecarboxylic acid or cyclopropanecarboxylic acid ethyl.

In another embodiment, provided is a compound according to formula (I), wherein R₃ is cyclobutyl, oxetanyl or unsubstituted lower alkyl.

In another embodiment, provided is a compound according to formula (I), wherein R₃ is lower alkyl substituted with phenyl substituted with —F, —Cl or —CF₃.

In another embodiment, provided is a compound according to formula (I), wherein X is oxygen; R₁ is lower alkyl; R₂ is phenyl substituted with halogen, —CH₂C(O)OH, cyclopropanecarboxylic acid, cyclopropanecarboxylic acid ethyl ester, methanesulfonylaminocarbonyl or tetrazole; and R₃ is lower alkyl substituted with phenyl substituted with halogen or —CF₃, or a pharmaceutically acceptable salt thereof.

In still another embodiment, provided is a compound according to formula (I), wherein X is oxygen; R₁ is methyl; R₂ is phenyl substituted with cyclopropanecarboxylic acid; and R₃ is lower alkyl substituted with phenyl substituted with halogen or —CF₃, or a pharmaceutically acceptable salt thereof.

Particular compounds of formula (I) include the following:
2-Methyl-4-phenyl-2H-pyrazol-3-yl-carbamic acid (R)-1-phenyl-ethyl ester;
{4′-[1-Methyl-5-((R)-1-phenyl-ethoxycarbonylamino)-1H-pyrazol-4-yl]-biphenyl-4-yl}-acetic acid;
(2-Methyl-4-phenyl-2H-pyrazol-3-yl)-carbamic acid 1-(2-chloro-phenyl)-ethyl ester;
1-{4′-[1-Methyl-5-((R)-1-phenyl-ethoxycarbonylamino)-1H-pyrazol-4-yl]-biphenyl-4-yl}-cyclopropanecarboxylic acid ethyl ester;
1-{4′-[1-Methyl-5-((R)-1-phenyl-ethoxycarbonylamino)-1H-pyrazol-4-yl]-biphenyl-4-yl}-cyclopropanecarboxylic acid;
1-(4′-{5-[1-(2-Chloro-phenyl)-ethoxycarbonylamino]-1-methyl-1H-pyrazol-4-yl}-biphenyl-4-yl)-cyclopropanecarboxylic acid;
(4-Biphenyl-4-yl-2-methyl-2H-pyrazol-3-yl)-carbamic acid (R)-1-phenyl-ethyl ester;
[4-(4-Methoxy-phenyl)-2-methyl-2H-pyrazol-3-yl]-carbamic acid (R)-1-phenyl-ethyl ester;
1-{4-[1-Methyl-5-((R)-1-phenyl-ethoxycarbonylamino)-1H-pyrazol-4-yl]-phenyl}-cyclopropanecarboxylic acid;
{4-[1-Methyl-5-((R)-1-phenyl-ethoxycarbonylamino)-1H-pyrazol-4-yl]-phenyl}-acetic acid;
[4-(4-Fluoro-phenyl)-2-methyl-2H-pyrazol-3-yl]-carbamic acid 1-(2-chloro-phenyl)-ethyl ester;
[4-(2-Fluoro-phenyl)-2-methyl-2H-pyrazol-3-yl]-carbamic acid 1-(3-trifluoromethyl-phenyl)-ethyl ester;
[4-(2-Fluoro-phenyl)-2-methyl-2H-pyrazol-3-yl]-carbamic acid 1-(2-chloro-phenyl)-ethyl ester;
(R)-1-{4′-[5-(sec-Butoxycarbonylamino)-1-methyl-1H-pyrazol-4-yl]-biphenyl-4-yl}-cyclopropanecarboxylic acid;
(R)-1-{4′-[5-(1,2-Dimethyl-propoxycarbonylamino)-1-methyl-1H-pyrazol-4-yl]-biphenyl-4-yl}-cyclopropanecarboxylic acid;
(R)-1-{4′-[5-((1-(2-Fluorophenyl)ethoxy)carbonylamino)-1-methyl-1H-pyrazol-4-yl]-biphenyl-4-yl}-cyclopropanecarboxylic acid;
(R)-1-{4′-[1-Methyl-5-((1-(3-(trifluoromethyl)phenyl)ethoxy)carbonylamino)-1H-pyrazol-4-yl]-biphenyl-4-yl}-cyclopropanecarboxylic acid;
1-{4′-[5-((1-(4-fluorophenyl)ethoxy)carbonylamino)-1-methyl-1H-pyrazol-4-yl]-biphenyl-4-yl}-cyclopropanecarboxylic acid;
1-{4′-[5-(cyclobutoxycarbonylamino)-1-methyl-1H-pyrazol-4-yl]-biphenyl-4-yl}-cyclopropanecarboxylic acid;
1-{4′-[1-Methyl-5-((oxetan-3-yloxy)carbonylamino)-1H-pyrazol-4-yl]-biphenyl-4-yl}-cyclopropanecarboxylic acid;
[4-(2-Fluoro-phenyl)-2-methyl-2H-pyrazol-3-yl]-carbamic acid (R)-1-(2-chloro-phenyl)-ethyl ester;
[4-(2-Fluoro-phenyl)-2-methyl-2H-pyrazol-3-yl]-carbamic acid (S)-1-(2-chloro-phenyl)-ethyl ester;
[4-(2-Fluoro-phenyl)-2-methyl-2H-pyrazol-3-yl]-carbamic acid (R)-1-(3-trifluoromethyl-phenyl)-ethyl ester;
1-{4′-[5-(3-Benzyl-ureido)-1-methyl-1H-pyrazol-4-yl]-biphenyl-4-yl}-cyclopropanecarboxylic acid; and
1-{4′-[1-Methyl-5-((S)-3-phenyl-butyrylamino)-1H-pyrazol-4-yl]-biphenyl-4-yl}-cyclopropanecarboxylic acid.

In another embodiment of the invention, provided is a compound of formula (I) for use as a therapeutically active substance.

In another embodiment of the invention, provided is a pharmaceutical composition comprising a therapeutically effective amount of a compound of formula (I) and a therapeutically inert carrier.

In another embodiment of the invention, provided is a use of a compound according to formula (I) for the treatment or prophylaxis of pulmonary fibrosis.

In another embodiment of the invention, provided is a use of a compound according to formula (I) for the preparation of a medicament for the treatment or prophylaxis of pulmonary fibrosis.

In another embodiment of the invention, provided is a compound according to formula (I) for the treatment or prophylaxis of pulmonary fibrosis.

In another embodiment of the invention, provided is compound according formula (I), when manufactured according to a process below.

In another embodiment of the invention, provided is a method for the treatment or prophylaxis of pulmonary fibrosis, which method comprises the step of administering a therapeutically effective amount of a compound of formula (I) to a patient in need thereof.

In another embodiment of the invention, provided is an invention as hereinbefore described.

It will be appreciated, that the compounds of general formula I in this invention may be derivatized at functional groups to provide derivatives which are capable of conversion back to the parent compound in vivo. Physiologically acceptable and metabolically labile derivatives, which are capable of producing the parent compounds of general formula I in vivo are also within the scope of this invention.

Compounds of the present invention can be prepared beginning with commercially available starting materials, or utilizing general synthetic techniques and procedures known to those skilled in the art. Chemicals may be purchased from companies such as for example Aldrich, Argonaut Technologies, VWR, Lancaster, Princeton, Alfa, Oakwood, TCI, Fluorochem, Apollo, Matrix, Maybridge or Meinoah. Chromatography supplies and equipment may be purchased from such companies as for example AnaLogix, Inc, Burlington, Wis.; Biotage AB, Charlottes-ville, VA; Analytical Sales and Services, Inc., Pompton Plains, N.J.; Teledyne Isco, Lincoln, Nebr.; VWR International, Bridgeport, N.J.; Varian Inc., Palo Alto, Calif., and Multigram II Mettler Toledo Instrument Newark, Del. Biotage, ISCO and Analogix columns are pre-packed silica gel columns used in standard chromatography. Final compounds and intermediates were named using the AutoNom2000 feature in the MDL ISIS Draw application.

The present invention is also directed to the administration of a therapeutically effective amount of a compound of formula I in combination or association with other drugs or active agents for the treatment of inflammatory or allergic diseases and disorders. In one embodiment, the present invention relates to a method for the treatment and/or prevention of such diseases or disorders comprising administering to a human or animal simultaneously, sequentially, or separately, a therapeutically effective amount of a compound of formula I and another drug or active agent (such as another anti-inflammatory or anti-allergic drug or agent). These other drugs or active agents may have the same, similar, or a completely different mode of action. Suitable other drugs or active agents may include, but are not limited to: Beta2-adrenergic agonists such as albuterol or salmeterol; corticosteroids such as dexamethasone or fluticasone; antihistamines such as loratidine; leukotriene antagonists such as montelukast or zafirlukast; anti-IgE antibody therapies such as omalizumab; anti-infectives such as fusidic acid (particularly for the treatment of atopic dermatitis); anti-fungals such as clotrimazole (particularly for the treatment of atopic dermatitis); immunosuppressants such as tacrolimus and pimecrolimus; other antagonists of PGD2 acting at other receptors such as DP antagonists; inhibitors of phosphodiesterase type 4 such as cilomilast; drugs that modulate cytokine production such as inhibitors of TNF-alpha converting enzyme (TACE); drugs that modulate the activity of Th2 cytokines IL-4 and IL-5 such as blocking monoclonal antibodies and soluble receptors; PPAR-gamma agonists such as rosiglitazone; and 5-lipoxygenase inhibitors such as zileuton.

The compounds of the present invention can be prepared by any conventional means. Suitable processes for synthesizing these compounds are provided in the examples. Generally, compounds of formula I can be prepared according to the schemes illustrated below. For example, certain compounds of the invention may be made using the approach outlined in Scheme 1.

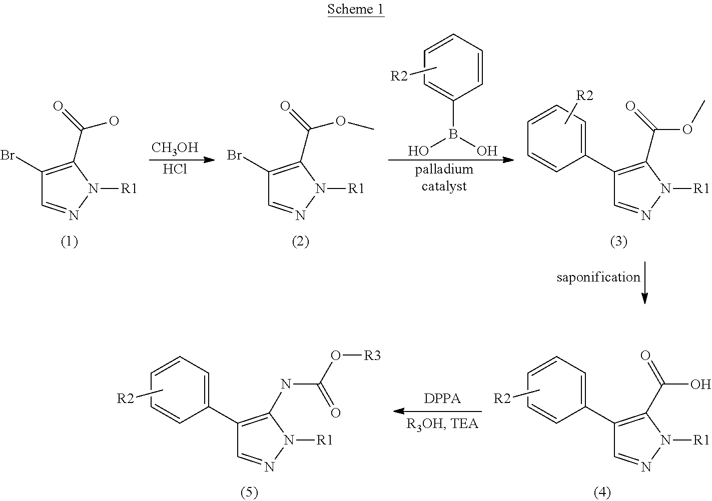

As described in Scheme 1, the bromo-substituted N-alkylpyrazole carboxylic acid (1), where R1 can be a lower alkyl group, can be esterified under acidic condition to provide the corresponding methyl ester (2). Compound (1) can be 4-bromo-2-methyl-2H-pyrazole-3-carboxylic acid. Under palladium catalyzed Suzuki coupling conditions, compound (3) can be formed through the reaction of compound (2) with the boronic acid, where R2 can be alkyl, aryl, halogen, and alkoxy groups. Hydrolysis of compound (3) under basic condition can provide the corresponding carboxylic acid (4), which can be converted to a carbamate (5) under Curtis rearrangement reaction conditions, where R3 can be alkyl, cycloalkyl or aryl-substituted alkyl groups.

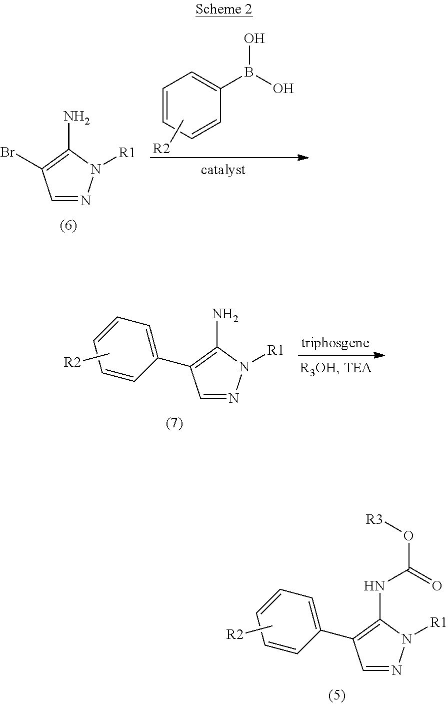

Alternatively, as described in Scheme 2, the bromo-substituted N-alkyl-aminopyrazole (6) can be coupled to arylboronic acid under palladium catalyst conditions to give compound (7), where R1 can be lower alkyl groups, such as methyl, and R2 can be alkyl, aryl, halogen and alkoxy groups. The aryl-substituted aminopyrazole intermediate (7) can react with triphosgene and substituted alcohols under basic condition to give a carbamate (5), where R3 can be alkyl, cycloalkyl or aryl-substituted alkyl groups.

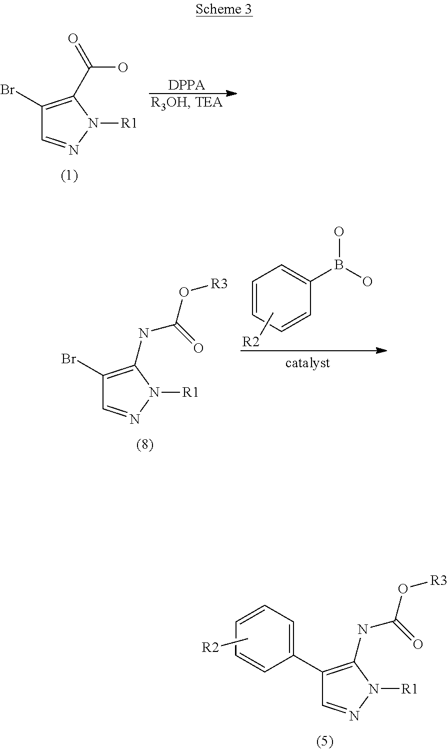

Alternatively, as described in Scheme 3, compound (1) can be reacted with substituted alcohols under Curtis rearrangement conditions to give the intermediate carbamate (8), where R1 can be lower alkyl groups, such as methyl group, and R3 can be alkyl, cycloalkyl or aryl-substituted alkyl groups. The coupling of compound (8) with arylboronic acid under palladium catalysis can provide the desired compound (5).

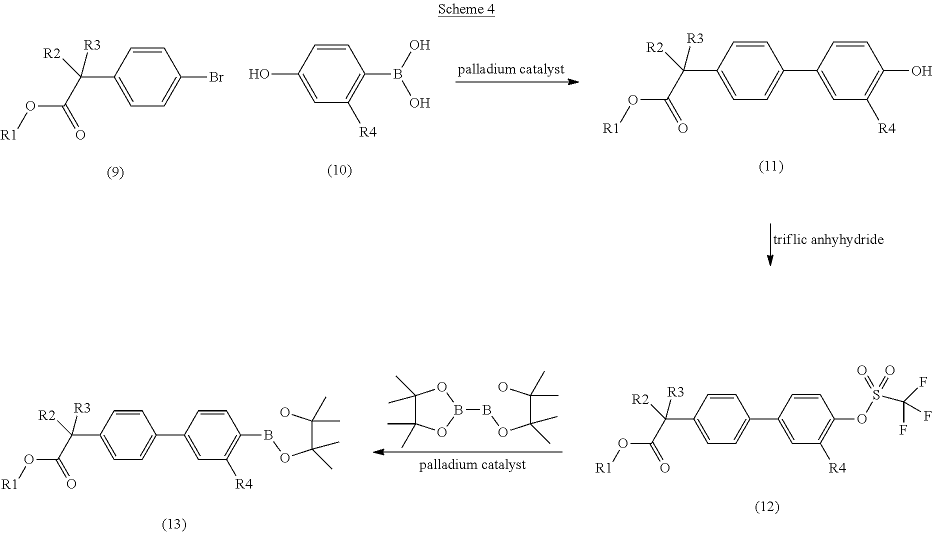

In case the required arylboronic acid is not commercially available, the preparation of the desired arylboronic acid is described in Scheme 4. The 4-bromophenylacetic acid derivatives (9) can react with 4-hydroxyboronic acid (10) under Suzuki coupling conditions, where R1 can be methyl or ethyl group, R2 and R3 can be hydrogen, lower alkyl groups, or R2 and R3 can be connected to form a ring, such as 3-membered, 4-membered or 5-membered carbocyclic rings, and R4 can be hydrogen, alkoxy, or halogen such as fluorine. The biarylphenol (11) can be converted to the corresponding triflate (12) through the reaction with triflic anhydride. Conversion of triflate in compound (12) into a cyclic boronate (13) can be accomplished through the reaction with bis-pinacolatodiborane under palladium catalysis.

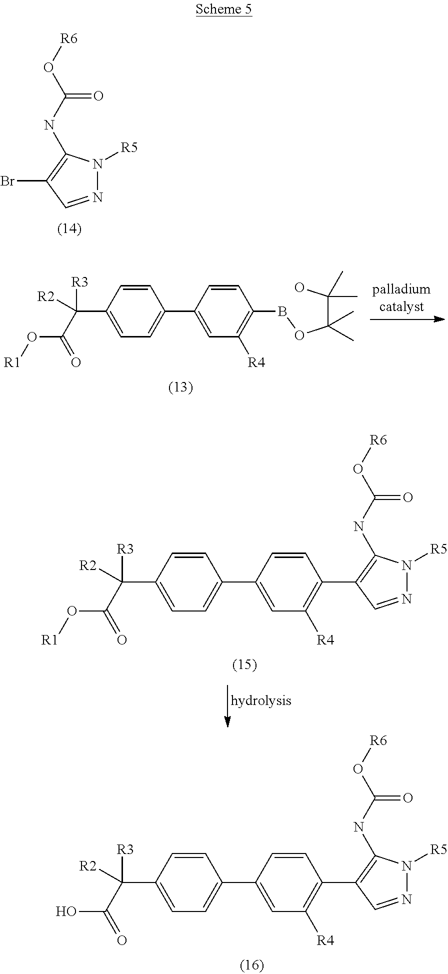

In order to prepare biaryl-substituted carboxylic acid derivatives (16) in Scheme 5, carbamate (14) can be coupled with the pinacolatoboronate (13) to provide the biaryl-substituted N-alkylpyrazole intermediate (15), where compound (14) can be the same structure as compound (8) described in Scheme 3. The palladium catalyst can be palladium acetate in the presence of phosphine ligand, such as X-Phos. Hydrolysis of compound (15) under basic condition can provide the desired carboxylic acid (16), where R2, R3 and R4 are defined in Scheme 4, R5 can be lower alkyl, such as methyl group, and R6 can be alkyl, cycloalkyl or aryl-substituted alkyl groups.

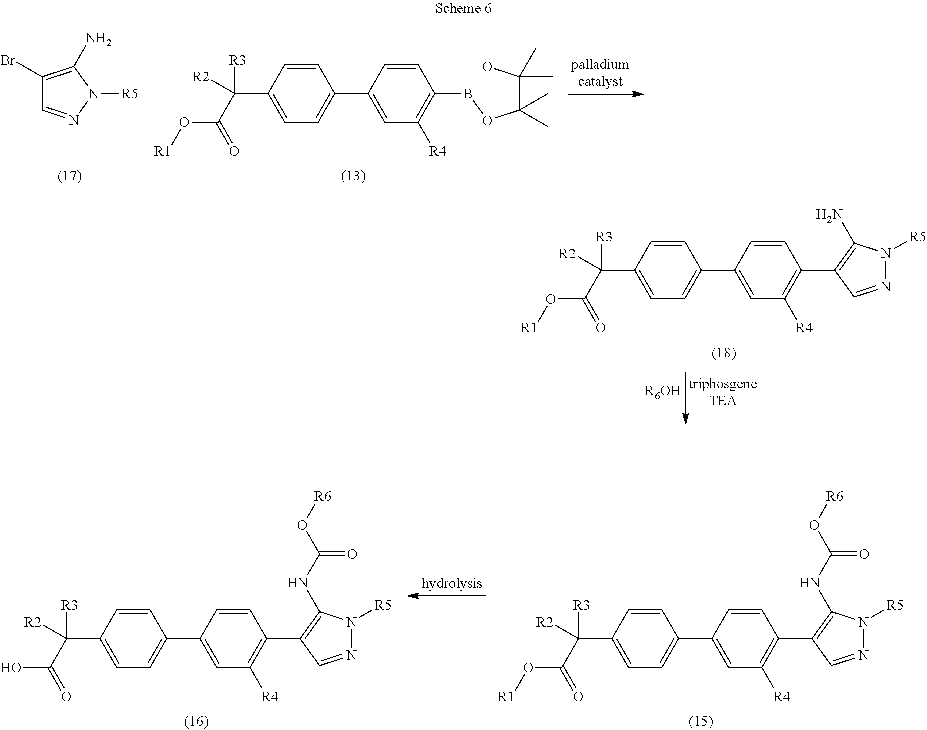

Alternatively, as described in Scheme 6, the bromo-substituted N-alkyl-aminopyrazole (17) can be coupled with biaryl-substituted pinacolatoborate (13) under palladium catalysis conditions to provide the biaryl-substituted aminopyrazole (18), where the structure (17) can be the same as the structure (6) in Scheme 2. The coupling condition can be palladium acetate in the presence of phosphine ligand, such as X-Phos. The biaryl-substituted aminopyrazole (18) can be derivatized to a corresponding carbamate (15) by reacting with substituted alcohol in the presence of triphosgene. Hydrolysis of ester (15) can provide the desired carboxylic acid (16).

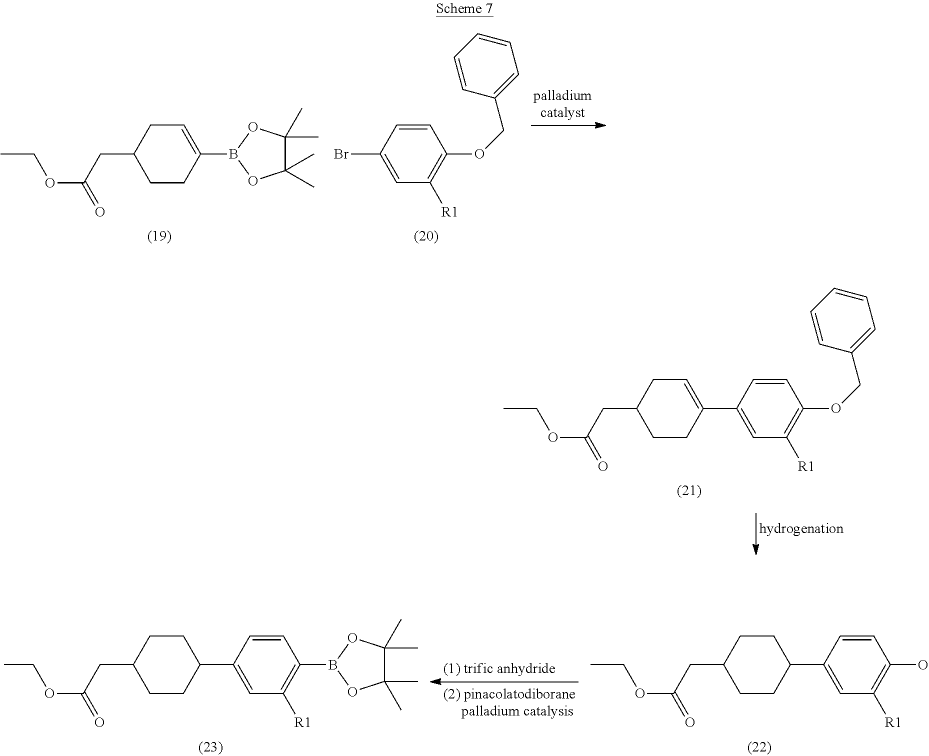

For the preparation of the cyclohexyl substituted arylboronic acid esters, the reaction is described in Scheme 7. Compound (19) can be prepared according to literature procedure (WO2009/016462). Compound (20) can be prepared from the corresponding bromophenol, where R1 can be hydrogen or fluorine. The coupling of compound (19) with compound (20) under palladium catalysis can provide compound (21). Hydrogenation of compound (21) can give the desired phenol (22), which can be converted to the corresponding pinacolatoborate (23). Compound (23) can be coupled to bromo-substituted aminopyrazole as described in Scheme 6 to provide the desired cyclohexyl substituted carboxylic acid.

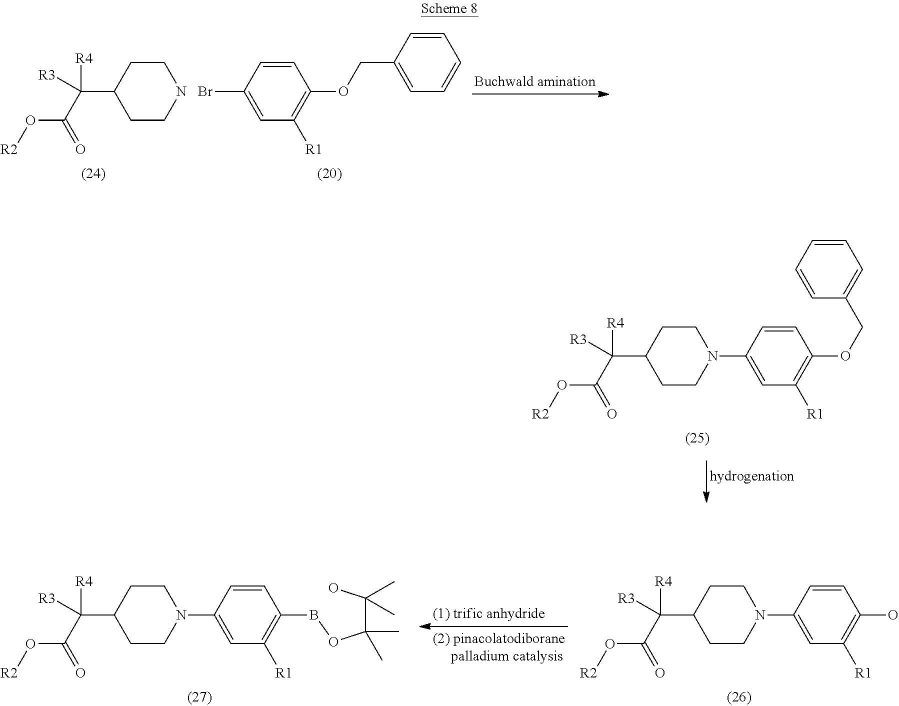

For the preparation of heterocycle substituted arylboronic acid, the reaction is described in Scheme 8. The piperidine acetic acid derivatives (24) can be commercially available or prepared according to literature. For the starting material (24), where R2 can be methyl, ethyl or tert-butyl, R3 and R4 can be hydrogen or alkyl, R3 and R4 can be connected to form a ring such as a three membered carbocyclic ring. For compound (24), where R3 and R4 is connected to form a cyclopropane ring, the preparation can be performed according to literature procedure (WO2008/053194). Under Buchwald/Hartwig amination conditions, the reaction of (24) with (20) can provide the heterocycle substituted arylphenol ether (25), where R1 can be hydrogen or fluorine. Hydrogenation of (25) followed by the conversion of phenol (26) to a pinacolatoboronate can provide the desired aryl boronic acid ester (27). Compound (27) can be coupled to bromo-substituted aminopyrazole as described in Scheme 6 to provide the desired heterocycle substituted carboxylic acid.

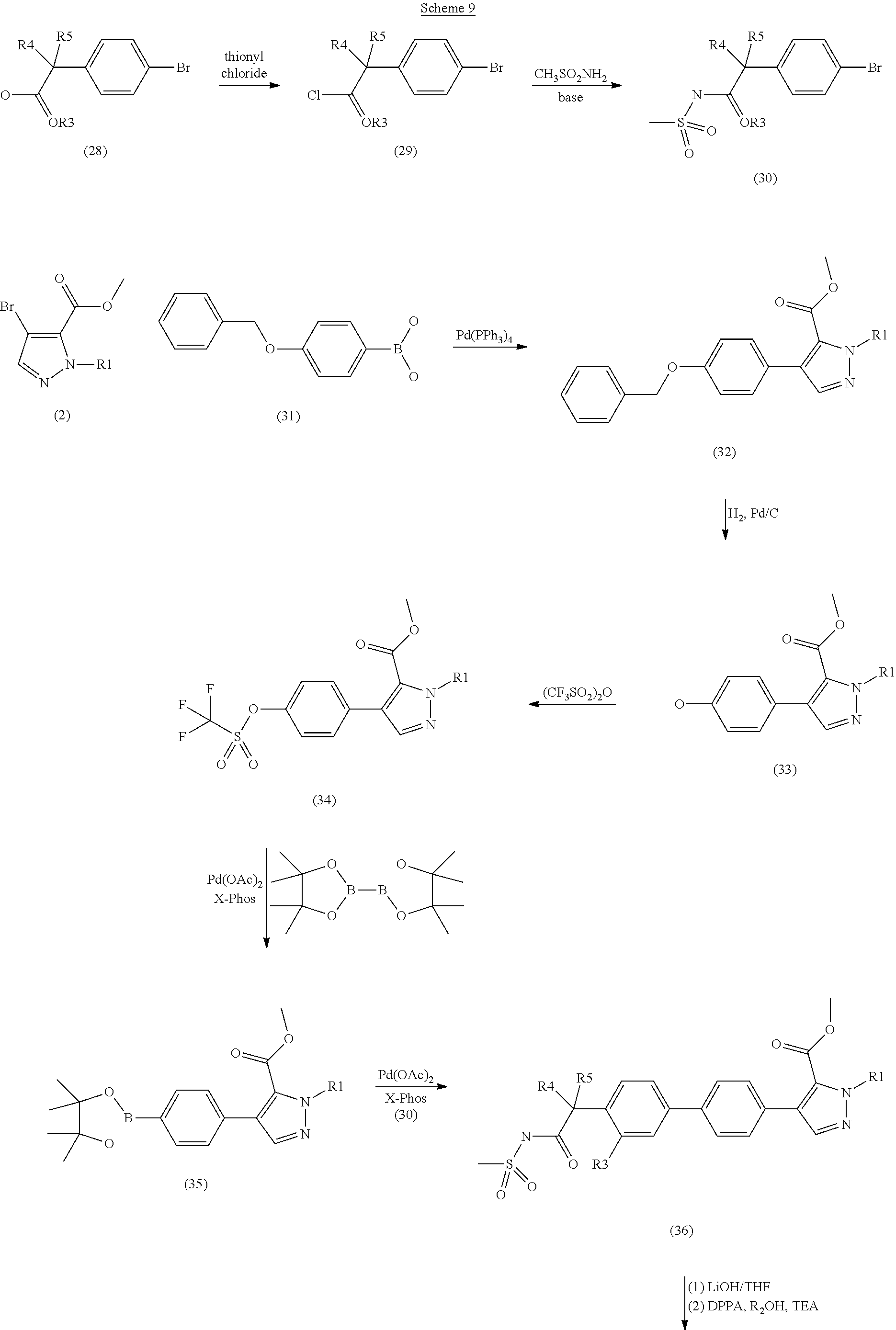

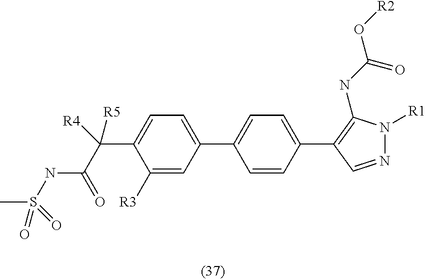

The preparation of carbonysulfonamide derivative (37) is described in Scheme 9. Commercially available carboxylic acid (28) can be converted to the corresponding acyl chloride (29) by reacting with thionyl chloride, where R3 in (28) can be hydrogen or fluorine, R4 and R5 can be connected to form a 3- or 4-membered carbon cyclic ring. Compound (29) can be converted to a carbonylsulfonamide derivative (30) by reacting with methanesulfonamide. The bromopyrazole derivative (2) can react with 4-benzyloxyphenylboronic acid (31) under Suzuki coupling condition to provide (32). Hydrogenation of (32) catalyzed by palladium can lead to a desired phenol derivative (33), which can be transformed to the corresponding triflate (34). Further reaction between triflate (34) and pinacolatodiborane under palladium catalysis condition can provide the key intermediate boronate derivative (35). The coupling between (35) and (30) under Suzuki coupling conditions can give the desired biphenyl derivative (36), which can be hydrolyzed under mild basic condition and further converted to the desired carbamate (37) under Curtius rearrangement conditions.

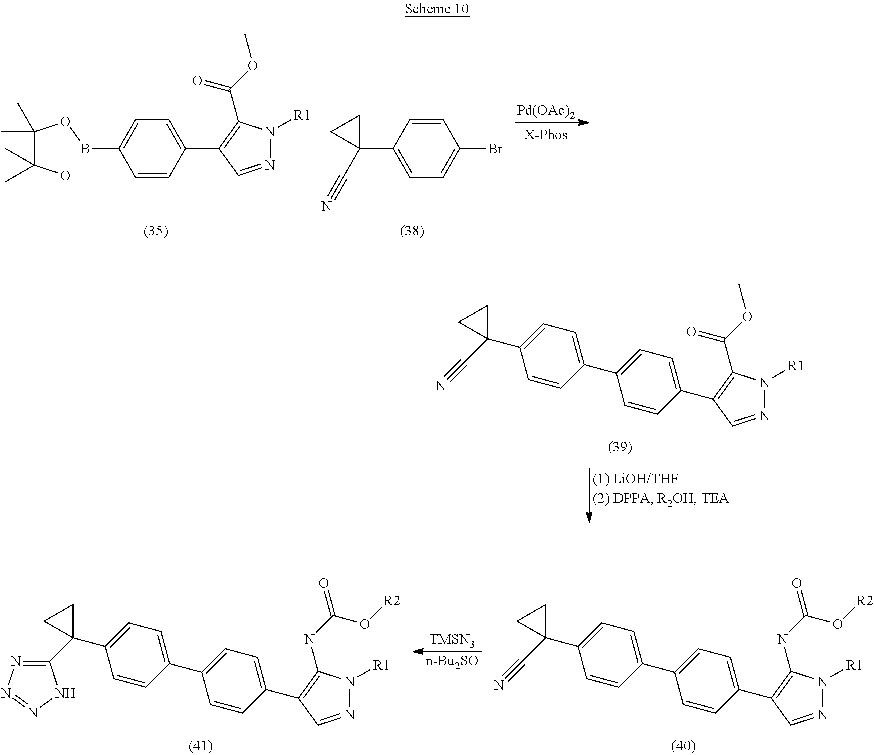

The preparation of tetrazole derivative (41) is described in Scheme 10. The reaction between boronate intermediate (35) and commercially available arylbromide (38) under Suzuki coupling condition can provide compound (39). Under mild basic conditions, compound (39) can be hydrolyzed to the corresponding carboxylic acid, which can undergo Curtius rearrangement to provide compound (40). Treatment of compound (40) with azidotrimethylsilane and di-n-butyltin oxide in heated toluene can lead to the desired tetrazole (41).

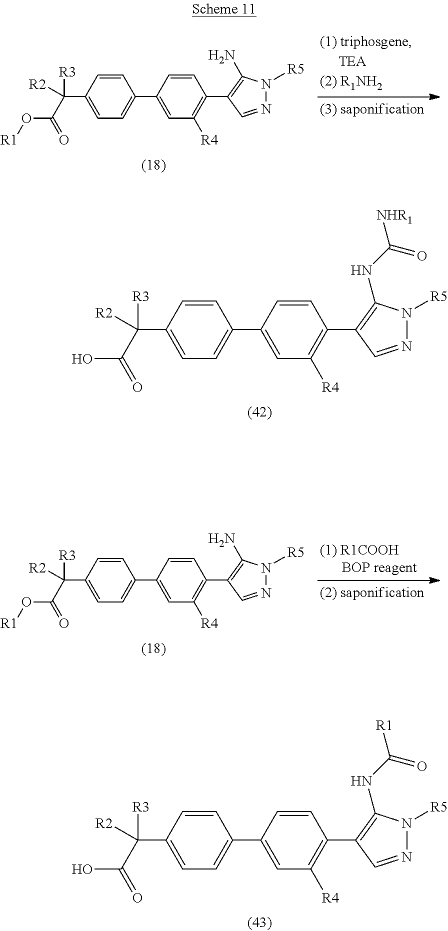

Finally, the preparation of urea (42) and carboxamide (43) is described in Scheme 11. The intermediate (18) from Scheme 6 can react with triphosgene and amine to provide the corresponding urea, which can be hydrolyzed to give the desired compound (42). With the same intermediate (18), carboxamide (43) can be obtained through the amide formation and ester hydrolysis.

### EXAMPLES

Although certain exemplary embodiments are depicted and described herein, the compounds of the present invention can be prepared using appropriate starting materials according to the methods described generally herein and/or by methods available to one of ordinary skill in the art.

Definition of abbreviations: DPPA: diphenylphosphorylazide; X-Phos: dicyclohexyl[2′,4′,6′-tris(1-methylethyl)[1,1′-biphenyl]-2-yl]-phosphine; S-Phos: dicyclohexyl(2′,6′-dimethoxy[1,1′-biphenyl]-2-yl)-phosphine; DMF: dimethylformamide; TEA: triethylamine. THF: tetrahydrofuran. TLC: thin layer chromatography. SFC: super critic fluid chromatography; ES+: electron spray positive charge; ES−: electron spray negative charge.

### Example 1

### 2-Methyl-4-phenyl-2H-pyrazol-3-yl-carbamic acid (R)-1-phenyl-ethyl ester

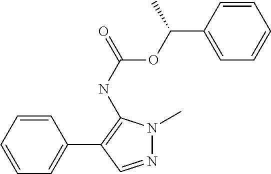 [SMILES: C[C@@H](OC(=O)Nc1c(-c2ccccc2)cnn1C)c1ccccc1]

4-Bromo-2-methyl-2H-pyrazole-3-carboxylic acid (6 g, 29.3 mmol) was added to 150 mL of methanol treated with thionyl chloride (3.5 g, 29.3 mmol). The mixture was refluxed for 18 hrs. Solvents were evaporated and the residue was extracted with dichloromethane and 0.5N sodium hydroxide solution. The organic layer was washed with brine and dried. After the evaporation of solvents, a white solid (4.19 g, 65.4% yield) was obtained as the desired compound 4-bromo-2-methyl-2H-pyrazole-3-carboxylic acid methyl ester. ¹H NMR (400 MHz, CDCl₃) δ ppm 3.97 (s, 3H), 4.19 (s, 3H), 7.51 (s, 1H). The aqueous extraction was filtered and neutralized with 1N hydrochloric acid. The white solid was filtered and dried to give the unreacted starting material carboxylic acid (1.51 g).

Methyl 4-bromo-2-methyl-2H-pyrazole-3-carboxylate (438.1 mg, 2.0 mmol), phenylboronic acid (244 mg, 2.0 mmol) and cesium carbonate (1.3 g, 4.0 mmol) were dissolved in DMF and the solution was degassed with argon. To this mixture was added Pd(PPh₃)₄ (139 mg, 0.12 mmol). The mixture was stirred at 80° C. for 12 hr. The resulting mixture was cooled to room temperature and filtered. The solid was rinsed with THF. The filtrate was concentrated and purified by ISCO flash column chromatography (0% to 25% ethyl acetate in hexanes, 40 g silica gel) to give an oily material as methyl 2-methyl-4-phenyl-2H-pyrazole-3-carboxylate (388.6 mg, 89.8% yield). ¹H NMR (400 MHz, CDCl₃) δ ppm 3.77 (s, 3H), 4.21 (s, 3H), 7.31-7.36 (m, 1H), 7.37-7.42 (m, 4H), 7.52 (s, 1H); LC/MS calcd for C₁₂H₁₂N₂O₂ 216.0, obsd 217.0 (M+H, ES+).

Methyl 2-methyl-4-phenyl-2H-pyrazole-3-carboxylate (388.6 mg, 1.8 mmol) was dissolved in THF (8 mL) and 0.5N LiOH solution (4 mL) was added. The mixture was stirred at 60° C. for 2 hrs and then concentrated. The residue was dissolved in water (30 mL) and filtered. The filtrate was neutralized with 1N hydrochloric acid and the white precipitate was filtered and dried in a vacuum oven at 60° C. overnight to provide 2-methyl-4-phenyl-2H-pyrazole-3-carboxylic acid (338.5 mg, 93.1% yield). ¹H NMR (300 MHz, DMSO-d₆) δ ppm 4.06 (s, 3H), 7.25-7.42 (m, 5H), 7.60 (s, 1H), 13.42 (s, 1H); LC/MS calcd for C₁₁H₁₀N₂O₂ 202.0, obsd 201.0 (M−H, ES−).

2-Methyl-4-phenyl-2H-pyrazole-3-carboxylic acid (100 mg, 0.495 mmol), (R)-1-phenylethanol (60.4 mg, 0.495 mmol), DPPA (136 mg, 0.495 mmol) and TEA (100 mg, 0.989 mmol) were mixed with 3 mL of toluene. The mixture was stirred at 80° C. for 1 hr. Solvents were evaporated and the residue was purified by ISCO flash column chromatography (40 g silica gel, 0% to 55% ethyl acetate in hexanes) to give 2-methyl-4-phenyl-2H-pyrazol-3-yl-carbamic acid (R)-1-phenyl-ethyl ester as a white powder (106 mg, 66.7% yield). ¹H NMR (300 MHz, DMSO-d₆) δ ppm 1.13-1.29 (br, 0.7 H), 1.52 (d, J=5.8 Hz, 2.3 H), 3.60 (s, 3H), 5.58-5.82 (br m, 1H), 6.92-7.53 (m, 10H), 7.74 (s, 1H), 9.18 (br, 0.2H), 9.55 (s, 0.8H); LC/MS calcd for C₁₉H₁₉N₃O₂ 321.0, obsd 320.0 (M−H, ES−).

### Example 2

### {4′-[1-Methyl-5-((R)-1-phenyl-ethoxycarbonylamino)-1H-pyrazol-4-yl]-biphenyl-4-yl}-acetic acid

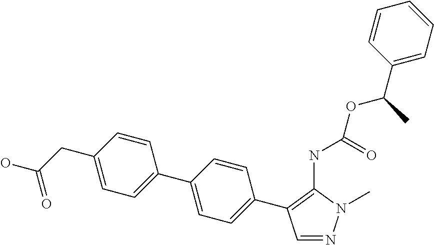 [SMILES: C[C@@H](OC(=O)Nc1c(-c2ccc(-c3ccc(CC(=O)O)cc3)cc2)cnn1C)c1ccccc1]

4-Bromo-2-methyl-2H-pyrazole-3-carboxylic acid (787.2 mg, 3.84 mmol), DPPA (1.16 g, 4.22 mmol), (R)-1-phenylethanol (491 mg, 4.02 mmol) and TEA (1.10 mL, 7.68 mmol) were combined in 15 mL of toluene to give a clear solution. The mixture was heated to 80° C. and stirred for 1 hr. Solvents were evaporated and the residue was extracted with ethyl acetate and sodium bicarbonate solution. The organic layer was dried and evaporated to give an oily material (1.38 g). TLC indicated no UV absorption. ¹H-NMR of the crude material indicated 75% desired compound as 4-bromo-2-methyl-2H-pyrazol-3-yl-carbamic acid (R)-1-phenyl-ethyl ester. LRMS calcd for C₁₃H₁₄BrN₃O₂ (m/e) 324.0, obsd 323.0 (M−H, ES−).

Ethyl 2-(4-bromophenyl)-acetate (2.43 g, 10 mmol), 4-hydroxyphenylboronic acid (1.65 g, 1.20 eq), Pd(PPh₃)₄ (693 mg, 0.06 eq) and potassium carbonate (2.76 g, 2.0 eq) were combined in 14 mL of dry DMF. The mixture was bubbled with nitrogen and sealed. The mixture was stirred at 85° C. for 15 hrs. Solvents were evaporated and the residue was extracted with ethyl acetate and water. The organic layer was washed with brine and dried over sodium sulfate. Solvents were evaporated and the residue was purified by ISCO flash column chromatography (5% to 50% ethyl acetate in hexanes). The pure fraction was combined and concentrated. The residue was dissolved in ethyl acetate (4 mL) and hot hexanes were added. The white solid was filtered and dried to give 4′-hydroxy-biphenyl-4-yl-acetic acid ethyl ester (1.68 g, 65.6% yield). ¹H NMR (400 MHz, CDCl₃) δ ppm 1.29 (t, J=7.1 Hz, 3H) 3.66 (s, 2H) 4.19 (q, J=7.1 Hz, 2H), 4.95 (br. s., 1H), 6.87 (d, J=8.6 Hz, 2H), 7.33 (d, J=8.3 Hz, 2H) 7.47 (m, 4H); LC/MS calcd for C₁₆H₁₆O₃ (m/e) 256.0, obsd 257.1 (M+H, ES+).

Ethyl 2-(4′-hydroxybiphenyl-4-yl)-acetate (641 mg, 2.5 mmol) was dissolved in dichloromethane (15 mL). To this solution was added trifluoromethanesulfonic anhydride (706 mg, 0.42 mL) at −78° C. Triethylamine (0.35 mL, 2.5 mmol) was added. The mixture was stirred at −78° C. for 10 minutes and warmed to room temperature. After 30 minutes, TLC indicated complete consumption of the starting material. The mixture was extracted with water and methylene chloride. The organic layer was washed with diluted hydrochloric acid and concentrated sodium bicarbonate solution. Solvents were evaporated and the residue was treated with hexanes. The grey crystalline material was filtered to give 4′-trifluoromethanesulfonyloxy-biphenyl-4-yl-acetic acid ethyl ester (812 mg, 83.6% yield). ¹H NMR (400 MHz, CDCl₃) δ ppm 1.29 (t, J=7.1 Hz, 3H), 3.68 (s, 2H), 4.19 (q, J=7.1 Hz, 2H), 7.35 (d, J=8.1 Hz, 2H), 7.39 (d, J=8.1 Hz, 2H), 7.53 (d, J=8.0 Hz, 2H), 7.64 (d, J=8.0 Hz, 2H).

Ethyl 2-(4′-(trifluoromethylsulfonyloxy)-biphenyl-4-yl)-acetate (800 mg, 2.06 mmol), 4,4,4′,4′,5,5,5′,5′-octamethyl-2,2′-bi(1,3,2-dioxaborolane) (628 mg, 2.47 mmol), potassium acetate (607 mg, 6.18 mmol) and Pd(dppf)Cl₂ (90.4 mg, 0.124 mmol) were combined in dry dioxane (15 mL). The mixture was stirred with argon bubbled through for 5 minutes. The mixture was heated to 90° C. and stirred for 4 hrs. TLC showed the same R_{f} as the starting material. LC/MS showed complete consumption of the starting material and the formation of the desired compound. The mixture was filtered through a layer of silica gel and rinsed with ethyl acetate. Solvents were evaporated and the residue was purified by ISCO flash column chromatography (40 g silica gel, 0% to 30% ethyl acetate in hexanes) to give [4′-(4,4,5,5-tetramethyl-[1,3,2]dioxaborolan-2-yl)-biphenyl-4-yl]-acetic acid ethyl ester as a white solid (728 mg, 96.5% yield). ¹H NMR (400 MHz, CDCl₃) δ ppm 1.28 (t, J=7.1 Hz, 3H), 1.37 (s, 12H), 3.67 (s, 2H), 4.18 (q, J=7.1 Hz, 2H), 7.37 (d, J=8.1 Hz, 2H), 7.60 (dd, J=8.0, 6.4 Hz, 4H), 7.88 (d, J=8.1 Hz, 2H).

Palladium acetate (12.5 mg, 0.055 mmol), X-PHOS (CAS#564483-18-7, 52.9 mg, 0.11 mmol) and potassium phosphate tribasic (236 mg, 1.11 mmol) were mixed in 0.5 mL of degassed water and 1 mL of toluene and stirred for 1 minute. Then 4-bromo-2-methyl-2H-pyrazol-3-yl-carbamic acid (R)-1-phenyl-ethyl ester (180 mg, 0.55 mmol) in 2 mL of toluene was added followed by the addition of [4′-(4,4,5,5-tetramethyl-[1,3,2]dioxaborolan-2-yl)-biphenyl-4-yl]-acetic acid ethyl ester (203 mg, 0.55 mmol) and 1 mL of toluene. The mixture was degassed with argon and sealed. The mixture was stirred at 95° C. overnight. The resulting mixture was extracted with ethyl acetate and water. The organic layer was dried and evaporated. The residue was purified by ISCO flash column chromatography (40 g silica gel, ethyl acetate with 5% methanol in hexanes) to give (R)-ethyl 2-(4′-(1-methyl-5-((1-phenylethoxy)carbonylamino)-1H-pyrazol-4-yl)-biphenyl-4-yl)-acetate (78.0 mg, 29.1% yield) as an amorphous material. LC/MS calcd for C₂₉H₂₉N₃O₄ (m/e) 483.0, obsd 484.1 (M+H, ES+).

(R)-ethyl 2-(4′-(1-methyl-5-((1-phenylethoxy)carbonylamino)-1H-pyrazol-4-yl)-biphenyl-4-yl)-acetate (78 mg, 0.161 mmol) was dissolved in 6 mL of THF and lithium hydroxide solution (1 mL, 0.5 N) was added followed by 0.2 mL of methanol. The mixture was stirred at room temperature for 4 hrs. TLC indicated complete consumption of the starting material. The mixture was concentrated and then dissolved in warm water (35 mL). The mixture was stirred and filtered. The filtrate was acidified with 1N hydrochloric acid (0.6 mL) and the mixture was filtered. The solid was dried under vacuum overnight to give a pale yellow solid as {4′-[1-methyl-5-((R)-1-phenyl-ethoxycarbonylamino)-1H-pyrazol-4-yl]-biphenyl-4-yl}-acetic acid (30 mg, 40.8% yield). ¹H NMR (400 MHz, DMSO-d₆) δ ppm 1.15-1.32 (br, 0.6H), 1.56 (d, J=5.8 Hz, 2.4H), 3.62 (s, 2H), 3.63 (s, 3H), 5.66-5.83 (br m, 1H), 7.03 (br, 0.5H), 7.18 (br, 0.5H), 7.28-7.38 (m, 3H), 7.38-7.48 (m, 3H), 7.49-7.56 (m, 1H), 7.57-7.68 (m, 5H), 7.81 (s, 1H), 9.24 (br, 0.2H), 9.62 (s, 0.8H), 12.38 (s, 1H); LC/MS calcd for C₂₇H₂₅N₃O₄ (m/e) 455.0, obsd 456.0 (M+H, ES+).

### Example 3

### (2-Methyl-4-phenyl-2H-pyrazol-3-yl)-carbamic acid 1-(2-chloro-phenyl)-ethyl ester

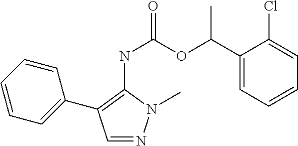 [SMILES: CC(OC(=O)Nc1c(-c2ccccc2)cnn1C)c1ccccc1Cl]

2-Methyl-4-phenyl-2H-pyrazole-3-carboxylic acid (prepared as the intermediate in Example 1, 60 mg, 0.297 mmol), 1-(2-chlorophenyl)ethanol (46.5 mg, 0.297 mmol), DPPA (81.7 mg, 0.297 mmol) and triethylamine (0.09 mL) were combined in 2.5 mL of toluene. The mixture was stirred at 85° C. for 3 hrs. Solvents were evaporated and the residue was purified by ISCO flash column chromatography (0% to 50% ethyl acetate in hexanes) to give (2-methyl-4-phenyl-2H-pyrazol-3-yl)-carbamic acid 1-(2-chloro-phenyl)-ethyl ester as a white fluffy solid (40 mg, 38% yield). ¹H NMR (400 MHz, DMSO-d₆) δ ppm 1.15-1.28 (br, 0.6H), 1.55 (d, J=5.8 Hz, 2.4H), 3.54-3.78 (m, 3H), 5.82-6.10 (m, 1H), 7.23 (d, J=6.8 Hz, 1H), 7.28-7.54 (m, 7H), 7.59 (d, J=6.8 Hz, 1H), 7.76 (br s, 1H), 9.31 (br, 0.2H), 9.68 (s, 0.8H). LC/MS calcd for C₁₉H₁₈ClN₃O₂ (m/e) 355.0, obsd 356.0 (M+H, ES+).

### Example 4

### 1-{4′-[1-Methyl-5-((R)-1-phenyl-ethoxycarbonylamino)-1H-pyrazol-4-yl]-biphenyl-4-yl}-cyclopropanecarboxylic acid ethyl ester

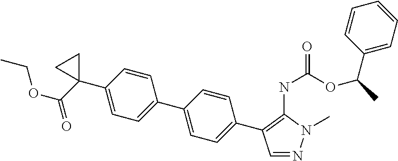 [SMILES: CCOC(=O)C1(c2ccc(-c3ccc(-c4cnn(C)c4NC(=O)O[C@H](C)c4ccccc4)cc3)cc2)CC1]

Ethyl 1-(4-bromophenyl)cyclopropanecarboxylate (2.5 g, 9.29 mmol), 4-hydroxyphenylboronic acid (1.67 g, 12.1 mmol), potassium carbonate (2.57 g, 18.6 mmol) and Pd(PPh₃)₄ (644 mg, 0.557 mmol) were combined in DMF (15 mL). The mixture was degassed with nitrogen and sealed. The mixture was stirred at 85° C. for 15 hrs. Solvents were evaporated and the residue was extracted with ethyl acetate and water. The organic layer was washed with diluted hydrochloric acid and water, dried over sodium sulfate and filtered. Solvents were evaporated and the residue was crystallized from ethyl acetate and hexanes to obtain the first batch light yellow solid (1.36 g) as ethyl 1-(4′-hydroxybiphenyl-4-yl)cyclopropanecarboxylate. The mother liquor was concentrated and crystallized from ethyl acetate and hexanes to give second batch of crystalline compound (330 mg). Both batches gave the same ¹H-NMR (total 1.69 g, 64.4% yield). ¹H-NMR (400 MHz, CDCl₃) δ ppm 1.15-1.32 (m, 5H), 1.60-1.71 (m, 2H), 4.14 (q, J=7.1 Hz, 2H), 5.15 (br s, 1H), 6.85 (d, J=8.6 Hz, 2H), 7.39 (d, J=8.1 Hz, 2H), 7.42-7.54 (m, 4H).

Ethyl 1-(4′-hydroxybiphenyl-4-yl)cyclopropanecarboxylate (1.41 g, 4.99 mmol) and TEA (0.8 mL) were dissolved in methylene chloride (80 mL). The solution was stirred under dry ice/acetone condition and trifluoromethanesulfonic anhydride (1.48 g, 5.24 mmol) in methylene chloride (4 mL) was added through a syringe. The solution was stirred for 30 minutes and cool bath was removed. The mixture was stirred at room temperature for 1 hr. The solution was extracted with methylene chloride and water. The organic layer was washed with diluted hydrochloric acid, water and sodium bicarbonate solution, dried over sodium sulfate and filtered. Solvents were evaporated to give an oily material (1.98 g, 95.7% yield) as ethyl 1-(4′-(trifluoromethylsulfonyloxy)-biphenyl-4-yl)-cyclopropanecarboxylate. ¹H-NMR (400 MHz, CDCl₃) δ ppm 1.16-1.26 (m, 5H), 1.62-1.70 (m, 2H), 4.13 (q, J=7.1 Hz, 2H), 7.35 (d, J=8.6 Hz, 2H), 7.44 (d, J=8.1 Hz, 2H), 7.51 (d, J=8.3 Hz, 2H), 7.65 (d, J=8.8 Hz, 2H).

Ethyl 1-(4′-(trifluoromethylsulfonyloxy)-biphenyl-4-yl)-cyclopropanecarboxylate (1.98 g, 4.78 mmol), 4,4,4′,4′,5,5,5′,5′-octamethyl-2,2′-bi(1,3,2-dioxaborolane) (1.46 g, 5.73 mmol) and potassium acetate (1.41 g, 14.3 mmol) were mixed in dry dioxane (15 mL). To this mixture was added Pd(dppf)Cl₂ (280 mg, 0.38 mmol) and the mixture was degassed with nitrogen for 2 minutes. The mixture was sealed and stirred under oil bath pre-heated to 90° C. After 4 hrs stirring, LC/MS indicated the desired product and no more starting material. The mixture was cooled to room temperature and diluted with ethyl acetate (40 mL). The mixture was filtered through a thin layer of Celite. The filtrate was concentrated and the residue was treated with ethyl acetate (30 mL) and hexanes (90 mL). The mixture was filtered. The filtrate was concentrated and purified by ISCO flash column chromatography (ethyl acetate in hexanes 0% to 20% in 20 minutes, 120 g silica gel) to give a white solid as ethyl 1-(4′-(4,4,5,5-tetramethyl-1,3,2-dioxaborolan-2-yl)-biphenyl-4-yl)-cyclopropanecarboxylate (1.03 g, 55% yield). ¹H-NMR (400 MHz, CDCl₃) δ ppm 1.17-1.30 (m, 5H), 1.39 (s, 12H), 1.60-1.71 (m, 2H), 4.14 (q, J=7.1 Hz, 2H), 7.43 (d, J=8.1 Hz, 2H), 7.54-7.70 (m, 4H), 7.90 (d, J=8.0 Hz, 2H).

Ethyl 1-(4′-(4,4,5,5-tetramethyl-1,3,2-dioxaborolan-2-yl)-biphenyl-4-yl)-cyclopropanecarboxylate (100 mg, 0.255 mmol), 4-bromo-1-methyl-1H-pyrazol-5-amine (67.3 mg, 1.50 eq), X-PHOS (36.5 mg, 0.30 eq), potassium phosphate tribasic (162 mg, 3.0 eq) and palladium acetate (8.6 mg, 0.15 eq) were mixed in 4 mL of toluene. The mixture was stirred and degassed water (0.8 mL) was added. The mixture was degassed with nitrogen and sealed. The mixture was stirred at 100° C. overnight and then extracted with ethyl acetate and water. Solvents were evaporated and the residue was purified by ISCO flash column chromatography (methanol in methylene chloride 0% to 10%, 12 g silica gel) to give a pale grey solid as 1-[4′-(5-amino-1-methyl-1H-pyrazol-4-yl)-biphenyl-4-yl]-cyclopropanecarboxylic acid ethyl ester (48 mg, 52.1% yield). ¹H-NMR (400 MHz, CDCl₃) δ ppm 1.19-1.25 (m, 5H), 1.64 (m, 2H), 3.77 (s, 3H), 3.81 (br s, 2H), 4.13 (q, J=7.1 Hz, 2H), 7.44 (t, J=8.8 Hz, 4H), 7.53 (s, 1H), 7.56 (d, J=8.1 Hz, 2H), 7.64 (d, J=8.1 Hz, 2H); LC/MS calcd for C₂₂H₂₃N₃O₂ (m/e) 361.0, obsd 362.1 (M+H, ES+).

Ethyl 1-(4′-(5-amino-1-methyl-1H-pyrazol-4-yl)-biphenyl-4-yl)-cyclopropanecarboxylate (100 mg, 0.277 mmol) and triphosgene (123 mg, 0.415 mmol) were dissolved in methylene chloride (2 mL) to give a solution. Toluene (6 mL) was added and the mixture was stirred followed by the addition of TEA (0.16 mL). The mixture was sealed and stirred at 90° C. for 10 minutes. (R)-(+)-1-Phenylethanol (68 mg, 0.553 mmol) in toluene (2 mL) was added. The mixture was stirred at 105° C. for 2 hrs. TLC indicated one major spot and complete disappearance of the starting material. The mixture was extracted with ethyl acetate and ammonium chloride solution. The organic layer was dried over sodium sulfate and filtered. Solvents were evaporated and the residue was purified by ISCO flash column chromatography (12 g silica gel, 0% to 60% ethyl acetate in hexanes in 15 minutes) to give a grey powder as 1-{4′-[1-methyl-5-((R)-1-phenyl-ethoxycarbonylamino)-1H-pyrazol-4-yl]-biphenyl-4-yl}-cyclopropanecarboxylic acid ethyl ester (101 mg, 71.6% yield). ¹H-NMR (400 MHz, CDCl₃) δ ppm 1.20-1.34 (m, 6H), 1.49-1.75 (m, 4H), 3.80 (s, 3H), 4.14 (q, J=7.1 Hz, 2H), 5.93 (m, 1H), 6.25 (br s, 1H), 7.35-7.48 (m, 9H), 7.53-7.64 (m, 4H), 7.72 (s, 1H); LC/MS calcd for C₃₁H₃₁N₃O₄ (m/e) 509.0, obsd 510.0 (M+H, ES+).

### Example 5

### 1-{4′-[1-Methyl-5-((R)-1-phenyl-ethoxycarbonylamino)-1H-pyrazol-4-yl]-biphenyl-4-yl}-cyclopropanecarboxylic acid

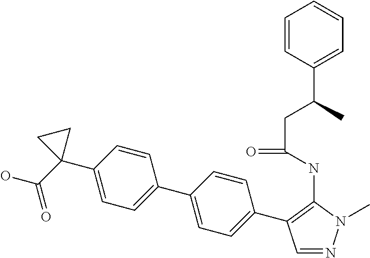 [SMILES: C[C@@H](CC(=O)Nc1c(-c2ccc(-c3ccc(C4(C(=O)O)CC4)cc3)cc2)cnn1C)c1ccccc1]

1-{4′-[1-Methyl-5-((R)-1-phenyl-ethoxycarbonylamino)-1H-pyrazol-4-yl]-biphenyl-4-yl}-cyclopropanecarboxylic acid ethyl ester (80 mg, 0.157 mmol) was dissolved in 4 mL of THF and lithium hydroxide solution (0.5N, 2 mL) was added. The mixture was stirred at room temperature for 5 minutes. Methanol (1 mL) was added to give a clear solution. The mixture was stirred at 65° C. for 5 hrs and then at 30° C. overnight. Solvents were evaporated and the residue was dissolved in water (12 mL). Hydrochloric acid (1N, 1.3 mL) was added and the solid was filtered to give a white solid (65 mg). LC/MS indicated 85% purity with the major impurity as the cleavage of carbamate. This solid was dissolved in acetonitrile/methylene chloride (containing 5% methanol) and purified by ISCO flash column chromatography (12 g silica gel, methanol in dichloromethane 0% to 5% in 15 minutes) to give a white solid as 1-{4′-[l-methyl-5-((R)-1-phenyl-ethoxycarbonylamino)-1H-pyrazol-4-yl]-biphenyl-4-yl}-cyclopropanecarboxylic acid (45 mg, 59.5% yield). ¹H-NMR (400 MHz, DMSO-d₆) δ ppm 1.19-1.25 (m, 2H), 1.49-1.57 (m, 5H), 3.64 (s, 3H), 5.65-5.83 (m, 1H), 6.95-7.25 (br m, 1H), 7.27-7.48 (m, 6H), 7.49-7.67 (m, 6H), 7.82 (br s, 1H), 9.25 (br s, 0.2H), 9.57 (s, 0.8H), 12.35 (s, 1H); LC/MS calcd for C₂₉H₂₇N₃O₄ (m/e) 481.0, obsd 482.1 (M+H, ES+).

### Example 6

### 1-(4′-{5-[1-(2-Chloro-phenyl)-ethoxycarbonylamino]-1-methyl-1H-pyrazol-4-yl}-biphenyl-4-yl)-cyclopropanecarboxylic acid

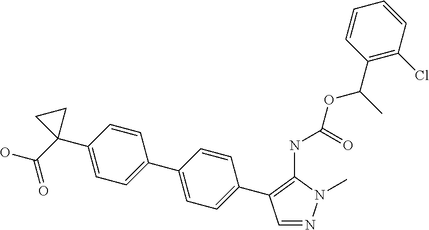 [SMILES: CC(OC(=O)Nc1c(-c2ccc(-c3ccc(C4(C(=O)O)CC4)cc3)cc2)cnn1C)c1ccccc1Cl]

Ethyl 1-(4′-(5-amino-1-methyl-1H-pyrazol-4-yl)-biphenyl-4-yl)-cyclopropanecarboxylate (intermediate from Example 4, 133 mg, 0.368 mmol) was mixed with triphosgene (164 mg, 0.552 mmol) in dichloromethane (5 mL). Toluene (5 mL) was added and the mixture was stirred. To this mixture was added TEA (0.5 mL) and the reaction tube was sealed. The mixture was stirred at 90° C. for 10 minutes and cooled to 40° C. 1-(2-Chlorophenyl)-ethanol (115 mg, 0.736 mmol) in toluene (1 mL) was added. The mixture was stirred at 105° C. for 1 hr. The mixture was cooled to room temperature and evaporated under reduced pressure. The residue was extracted with ethyl acetate and water, dried and evaporated. The residue was purified by ISCO flash column chromatography (40 g silica gel, 10% to 65% ethyl acetate in hexanes) to give a white fluffy solid as 1-(4′-{5-[1-(2-chloro-phenyl)-ethoxycarbonylamino]-1-methyl-1H-pyrazol-4-yl}-biphenyl-4-yl)-cyclopropanecarboxylic acid ethyl ester (141 mg, 70.4% yield). LC/MS calcd for C₃₁H₃₀ClN₃O₄ (m/e) 543, obsd 544.1 (M+H, ES+).

1-(4′-{5-[1-(2-Chloro-phenyl)-ethoxycarbonylamino]-1-methyl-1H-pyrazol-4-yl}-biphenyl-4-yl)-cyclopropanecarboxylic acid ethyl ester (140 mg, 0.257 mmol) was dissolved in 4 mL of THF and LiOH solution (0.5N, 2 mL) was added. The mixture was stirred at 65° C. overnight. LC/MS indicated significant amount of the des-carbamate side product. The mixture was concentrated and treated with 1N hydrochloric acid. The white solid was filtered and dried (75 mg). This material was purified using reverse phase HPLC (acetonitrile in water) to give 1-(4′-{5-[1-(2-chloro-phenyl)-ethoxycarbonylamino]-1-methyl-1H-pyrazol-4-yl}-biphenyl-4-yl)-cyclopropanecarboxylic acid as a white lyophilized amorous material (20 mg, 15.1% yield). ¹H-NMR (400 MHz, DMSO-d₆) ppm 1.14-1.30 (m, 2.6H), 1.42-1.52 (m, 2H), 1.57 (d, J=5.8 Hz, 2.4H), 6.03 (d, J=6.1 Hz, 1H), 7.29-7.69 (m, 12H), 7.83 (s, 1H), 9.37 (br s, 0.2H), 9.74 (s, 0.8H), 12.35 (br s, 1H); LC/MS calcd for C₂₉H₂₆ClN₃O₄ (m/e) 515.0, obsd 516.0 (M+H, ES+).

### Example 7

### (4-Biphenyl-4-yl-2-methyl-2H-pyrazol-3-yl)-carbamic acid (R)-1-phenyl-ethyl ester

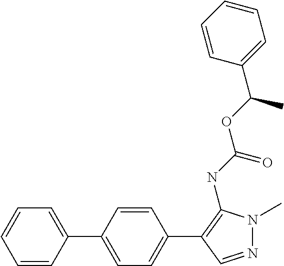 [SMILES: C[C@@H](OC(=O)Nc1c(-c2ccc(-c3ccccc3)cc2)cnn1C)c1ccccc1]

This compound was prepared with the same method as described for the preparation of 2-methyl-4-phenyl-2H-pyrazol-3-yl-carbamic acid (R)-1-phenyl-ethyl ester by using biphenyl-4-yl-boronic acid and 4-bromo-2-methyl-2H-pyrazol-3-carboxylic acid methyl ester. LC/MS calcd for C₂₅H₂₃N₃O₂ (m/e) 397, obsd 398.0 (M+H, ES+).

### Example 8

### [4-(4-Methoxy-phenyl)-2-methyl-2H-pyrazol-3-yl]-carbamic acid (R)-1-phenyl-ethyl ester

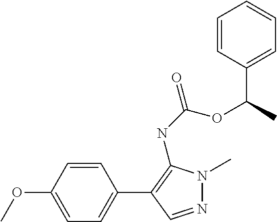 [SMILES: COc1ccc(-c2cnn(C)c2NC(=O)O[C@H](C)c2ccccc2)cc1]

This compound was prepared with the same method as described for the preparation of 2-methyl-4-phenyl-2H-pyrazol-3-yl-carbamic acid (R)-1-phenyl-ethyl ester by using 4-methoxyphenyl-boronic acid and 4-bromo-2-methyl-2H-pyrazol-3-carboxylic acid methyl ester. ¹H-NMR (400 MHz, DMSO-d₆) δ ppm 1.15-1.30 (br, 0.6H), 1.54 (d, J=5.8 Hz, 2.4 H), 3.60 (br s, 3H), 3.75 (s, 3H), 5.76 (d, J=6.1 Hz, 1H), 6.87 (d, J=7.6 Hz, 2H), 6.97-7.25 (m, 1H), 7.28-7.51 (m, 6H), 7.67 (s, 1H), 9.12 (br s, 0.2H), 9.48 (br s, 0.8H); LC/MS calcd for C₂₅H₂₃N₃O₂ (m/e) 397, obsd 398.0 (M+H, ES+); LC/MS calcd for C₂₀H₂₁N₃O₃ (m/e) 351, obsd 350.0 (M−H, ES−).

### Example 9

### 1-{4-[1-Methyl-5-((R)-1-phenyl-ethoxycarbonylamino)-1H-pyrazol-4-yl]-phenyl}-cyclopropanecarboxylic acid

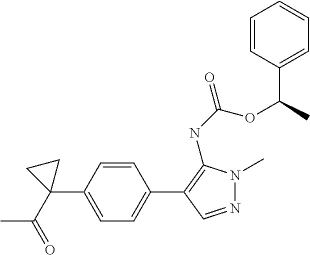 [SMILES: CC(=O)C1(c2ccc(-c3cnn(C)c3NC(=O)O[C@H](C)c3ccccc3)cc2)CC1]

(R)-1-phenylethyl 4-bromo-1-methyl-1H-pyrazol-5-yl-carbamate (165 mg, 0.51 mmol), methyl 1-(4-(4,4,5,5-tetramethyl-1,3,2-dioxaborolan-2-yl)-phenyl)-cyclopropanecarboxylate (154 mg, 0.51 mmol), S-PHOS (CAS#657408-07-6, 62.7 mg, 0.153 mmol), palladium acetate (17.1 mg, 0.076 mmol) were dissolved in toluene (4 mL) and potassium phosphate tribasic (324 mg, 1.53 mmol) in degassed water (1 mL) was added. The mixture was degassed for 5 minutes and sealed. The mixture was stirred at 100° C. overnight and then extracted with ethyl acetate and water. The organic layer was washed with brine and dried. Solvents were evaporated and the residue was purified by ISCO flash column chromatography using ethyl acetate in hexanes (0% to 50%, 24 g silica gel) to give a waxy pale yellow material as 1-{4-[1-methyl-5-((R)-1-phenyl-ethoxycarbonylamino)-1H-pyrazol-4-yl]-phenyl}-cyclopropanecarboxylic acid methyl ester (123 mg, 57.6% yield). LC/MS calcd for C₂₄H₂₅N₃O₄ (m/e) 419.0, obsd 420.0 (M+H, ES+).

(R)-methyl 1-(4-(1-methyl-5-((1-phenylethoxy)carbonylamino)-1H-pyrazol-4-yl)-phenyl)-cyclopropanecarboxylate (123 mg, 0.29 mmol) was dissolved in THF (4 mL) and aqueous lithium hydroxide solution (0.5N, 1 mL) was added. The reaction was heated to 65° C. and stirred for 4 hrs. Solvents were evaporated and the residue was treated with warm water (25 mL) and stirred at room temperature for 1 hr. The mixture was filtered and the filtrate was treated with 1N hydrochloric acid (0.5 mL). The white solid was filtered and rinsed with water and dried in air to give 1-{4-[1-methyl-5-((R)-1-phenyl-ethoxycarbonylamino)-1H-pyrazol-4-yl]-phenyl}-cyclopropanecarboxylic acid (42 mg, 35.4% yield). LC/MS calcd for C₂₃H₂₃N₃O₄ (m/e) 405, obsd 406 (M+H, ES+).

### Example 10

### {4-[1-Methyl-5-((R)-1-phenyl-ethoxycarbonylamino)-1H-pyrazol-4-yl]-phenyl}-acetic acid

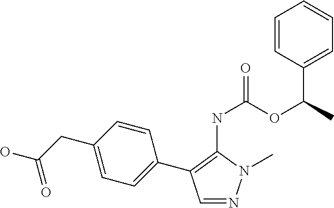 [SMILES: C[C@@H](OC(=O)Nc1c(-c2ccc(CC(=O)O)cc2)cnn1C)c1ccccc1]

This compound was prepared using the same method as described for the preparation of 1-{4-[1-methyl-5-((R)-1-phenyl-ethoxycarbonylamino)-1H-pyrazol-4-yl]-phenyl}-cyclopropanecarboxylic acid by using ethyl 2-(4-(4,4,5,5-tetramethyl-1,3,2-dioxaborolan-2-yl)-phenyl)-acetate and (R)-1-phenylethyl 4-bromo-1-methyl-1H-pyrazol-5-yl-carbamate. LC/MS calcd for C₂₁H₂₁N₃O₄ (m/e) 379.0, obsd 380.0 (M+H, ES+).

### Example 11

### [4-(4-Fluoro-phenyl)-2-methyl-2H-pyrazol-3-yl]-carbamic acid 1-(2-chloro-phenyl)-ethyl ester

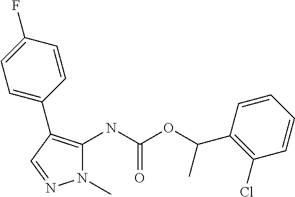 [SMILES: CC(OC(=O)Nc1c(-c2ccc(F)cc2)cnn1C)c1ccccc1Cl]

4-Fluorophenylboronic acid (211 mg, 1.51 mmol), methyl 4-bromo-1-methyl-1H-pyrazole-5-carboxylate (300 mg, 1.37 mmol), cesium carbonate (893 mg, 2.74 mmol) and Pd(PPh₃)₄ (95 mg, 0.082 mmol) were mixed in toluene (12 mL). The mixture was degassed with nitrogen and sealed. The mixture was stirred at 90° C. overnight and then concentrated. The residue was extracted with ethyl acetate and water. The organic layer was dried and evaporated. The residue was purified by ISCO flash column chromatography (40 g silica gel, 0% to 30% ethyl acetate in hexanes) to give an oily material as 4-(4-fluorophenyl)-2-methyl-2H-pyrazol-3-carboxylic acid methyl ester (140 mg, 43.6% yield). ¹H-NMR (400 MHz, CDCl₃) δ ppm 3.78 (s, 3H), 4.21 (s, 3H), 7.08 (t, J=8.5 Hz, 2H), 7.35 (m, 2H), 7.49 (s, 1H).

4-(4-Fluorophenyl)-2-methyl-2H-pyrazole-3-carboxylic acid methyl ester (140 mg, 0.60 mmol) was dissolved in THF (4 mL) and LiOH solution (0.5 N, 2 mL) was added. The mixture was stirred at room temperature for 4 hrs. The mixture was concentrated and dissolved in water. The solution was treated with 1N hydrochloric acid (1.10 mL). The mixture was extracted with ethyl acetate. The organic layer was dried and concentrated to give an oily material (115 mg, 87.4% yield) as 4-(4-fluorophenyl)-2-methyl-2H-pyrazole-3-carboxylic acid.

4-(4-Fluorophenyl)-2-methyl-2H-pyrazole-3-carboxylic acid (106 mg, 0.48 mmol), DPPA (132 mg, 0.48 mmol), 1-(2-chlorophenyl)ethanol (76 mg, 0.48 mmol) and TEA (0.2 mL) was stirred in 5 mL of toluene. The mixture was heated to 90° C. and stirred for 1 hr. Solvents were evaporated and the residue was extracted with ethyl acetate and water. The organic layer was washed with sodium bicarbonate solution and dried. Solvents were evaporated and the residue was purified by ISCO flash column chromatography (ethyl acetate in hexanes 0% to 60%) to give [4-(4-fluoro-phenyl)-2-methyl-2H-pyrazol-3-yl]-carbamic acid 1-(2-chloro-phenyl)-ethyl ester as a white solid (116 mg, 64.5% yield). ¹H-NMR (400 MHz, DMSO-d₆) δ ppm 1.15-1.30 (br, 0.6H), 1.56 (br d, J=5.3 Hz, 2.4H), 3.62 (br s, 3H), 5.86-6.06 (m, 1H), 6.81-7.27 (m, 3H), 7.31-7.65 (m, 5H), 7.76 (br s, 1H), 9.32 (s, 0.2H), 9.65 (s, 0.8H); LC/MS calcd for C₁₉H₁₇C1FN₃O₂ (m/e) 373.0, obsd 374.0 (M+H, ES+).

### Example 12

### [4-(2-Fluoro-phenyl)-2-methyl-2H-pyrazol-3-yl]-carbamic acid 1-(3-trifluoromethyl-phenyl)-ethyl ester

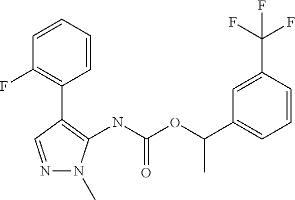 [SMILES: CC(OC(=O)Nc1c(-c2ccccc2F)cnn1C)c1cccc(C(F)(F)F)c1]

4-Bromo-1-methyl-1H-pyrazol-5-amine (800 mg, 4.55 mmol), 2-fluorophenylboronic acid (890 mg, 6.36 mmol), X-PHOS (217 mg, 0.45 mmol), palladium acetate (51 mg, 0.227 mmol) and potassium phosphate tribasic (1.93 g, 9.09 mmol) were combined and toluene (12 mL) was added. To the stirred mixture was added degassed water (4 mL) and the mixture was degassed and then sealed. The mixture was stirred at 95° C. overnight. Solvents were evaporated and the residue was extracted with ethyl acetate and water. The organic layer was washed with brine and dried. Solvents were evaporated and the residue was purified by ISCO flash column chromate-graphy (ethyl acetate containing 3% methanol in hexanes 10% to 80%, 40 g silica gel) to give 4-(2-fluorophenyl)-2-methyl-2H-pyrazole-3-amine as a brown oil (649 mg, 74.7% yield). ¹H-NMR (400 MHz, DMSO-d₆) δ ppm 3.60 (s, 3H), 5.30 (br s, 2H), 7.14-7.25 (m, 3H), 7.30 (d, J=2.5 Hz, 1H), 7.39-7.52 (m, 1H); LC/MS calcd for C₁₀H₁₀FN₃ (m/e) 191.0, obsd 192.0 (M+H, ES+).

4-(2-Fluorophenyl)-2-methyl-2H-pyrazol-3-amine (150 mg, 0.78 mmol) and triphosgene (303 mg, 1.02 mmol) were dissolved in dichloromethane (3 mL). Toluene (8 mL) was added and the mixture was sealed. The mixture was stirred in an ice bath and TEA (0.9 mL, 8.0 eq) was added. The mixture was stirred at 85° C. for 20 minutes and 1-(3-(trifluoromethyl)-phenyl)ethanol (194 mg, 1.02 mmol) in toluene (2 mL) was added. The mixture was stirred at 90° C. for 2 hr. Solvents were evaporated and the residue was extracted with ethyl acetate and water. The organic layer was washed with brine and dried. Solvents were evaporated and the residue was purified by ISCO flash column chromatography (0% to 60% ethyl acetate in hexanes) to give [4-(2-fluoro-phenyl)-2-methyl-2H-pyrazol-3-yl]-carbamic acid 1-(3-trifluoromethyl-phenyl)-ethyl ester as a pale yellow waxy material (226 mg, 70.7% yield). ¹H-NMR (400 MHz, DMSO-d₆) δ ppm 1.11-1.29 (m, 0.6H), 1.55 (br d, J=5.81 Hz, 2.4H), 3.65 (s, 3H), 5.73-5.85 (m, 1H), 7.06-7.24 (m, 2H), 7.25-7.52 (m, 3H), 7.56-7.82 (m, 4H), 9.29 (br s, 0.2H), 9.66 (s, 0.8H); LC/MS calcd for C₂₀H₁₇F₄N₃O₂ (m/e) 407.0, obsd 408.0 (M+H, ES+).

### Example 13

### [4-(2-Fluoro-phenyl)-2-methyl-2H-pyrazol-3-yl]-carbamic acid 1-(2-chloro-phenyl)-ethyl ester

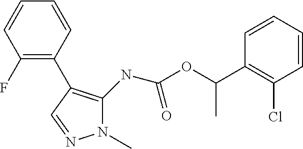 [SMILES: CC(OC(=O)Nc1c(-c2ccccc2F)cnn1C)c1ccccc1Cl]

4-(2-Fluorophenyl)-2-methyl-2H-pyrazol-3-amine (110 mg, 0.575 mmol) and triphosgene (222 mg, 0.748 mmol) were mixed in dichloromethane (4 mL) and toluene (6 mL) was added and the tube was sealed. The mixture was stirred in an ice bath and triethylamine (0.7 mL) was added. The mixture was stirred at 85° C. for 20 minutes and 1-(2-chlorophenyl)ethanol (117 mg, 0.748 mmol) in 1 mL of toluene was added. The mixture was stirred at 90° C. for 1 hr. Solvents were evaporated and the residue was extracted with ethyl acetate and water. The organic layer was dried and concentrated. The residue was purified by ISCO flash column chromatography (ethyl acetate in hexanes 0% to 50%) to give [4-(2-fluoro-phenyl)-2-methyl-2H-pyrazol-3-yl]-carbamic acid 1-(2-chloro-phenyl)-ethyl ester as amorphous powder (134 mg, 62.3% yield). ¹H-NMR (400 MHz, DMSO-d₆) δ ppm 1.06-1.28 (m, 0.6H), 1.53 (br d, J=5.6 Hz, 2.4H), 3.65 (br s, 3H), 5.83-5.98 (m, 1H), 6.83-7.50 (m, 7H), 7.56 (d, J=6.8 Hz, 1H), 7.65 (br s, 1H), 9.31 (br, 0.2H), 9.66 (s, 0.8H); LC/MS calcd for C₁₉H₁₇C1FN₃O₂ (m/e) 373.0, obsd 372.0 (M−H, ES−).

### Example 14

### (R)-1-{4′-[5-(sec-Butoxycarbonylamino)-1-methyl-1H-pyrazol-4-yl]-biphenyl-4-yl}-cyclopropanecarboxylic acid

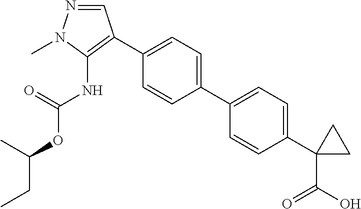 [SMILES: CC[C@@H](C)OC(=O)Nc1c(-c2ccc(-c3ccc(C4(C(=O)O)CC4)cc3)cc2)cnn1C]

In a 250 mL round-bottomed flask, methyl 1-(4-bromophenyl)-cyclopropanecarboxylate (9.4 g, 36.8 mmol), 4-hydroxyphenylboronic acid (6.61 g, 47.9 mmol, 1.3 eq) and potassium carbonate (10.2 g, 73.7 mmol, 2.0 eq) were combined with DMF (50 mL) to give a light brown suspension. Pd(Ph₃P)₄ (2.55 g, 2.21 mmol, 0.06 eq) was added and the mixture was evacuated and purged with argon. The reaction mixture was heated to 85° C. and stirred for 17 h under argon. The crude reaction mixture was concentrated in vacuo. The residue was partitioned between H₂O and EtOAc and filtered. The phases were separated and the organic layer was washed with 0.1 M HCl (15 mL), H₂O (15 mL) and sat NaCl (15 mL). The organic layer was dried over Na₂SO₄ and concentrated in vacuo. The residue was taken up in hot EtOAc and hexane and decanted from an insoluble red solid. The yellow supernatant was stripped and recrystallization from EtOAc and hexanes to afford 4.49 g (46%) of methyl 1-(4′-hydroxybiphenyl-4-yl)cyclopropanecarboxylate as an off white solid. The filtrate was stripped and the residue was recrystallized from EtOAc/hexane to afford an additional 1.64 g (17%) of the desired product as a pink powder. ¹H NMR (DMSO-d₆) δ ppm 9.53 (s, 1H), 7.45-7.55 (m, 4H), 7.29-7.38 (m, 2H), 6.82-6.86 (m, 2H), 3.59 (s, 3H),), 1.46-1.52 (m, 2H), 1.20-1.24 (m, 2H).

In a 500 ml, round-bottomed flask, methyl 1-(4′-hydroxybiphenyl-4-yl)cyclopropanecarboxylate (3 g, 11.2 mmol) and TEA (1.64 mL, 11.7 mmol, 1.05 eq) were combined with dichloromethane (200 mL) to give a yellow suspension. The mixture was cooled to −78° C. and triflic anhydride (3.31 g, 11.7 mmol, 1.05 eq) was added. The reaction was stirred at −78° C. for 30 mins, then at 25° C. for 1 h. The reaction mixture was diluted with H₂O and the organic layer was washed with 0.5M HCl (200 mL), H₂O (200 mL), and sat NaHCO₃ (150 mL). The organic layer was dried over Na₂SO₄ and filtered over a bed of silica gel to remove a dark red impurity. The filtrate was concentrated in vacuo to give 1-(4′-trifluoromethanesulfonyloxy-biphenyl-4-yl)cyclopropanecarboxylic acid methyl ester. The crude material was used without further purification in the subsequent reaction.

In a 1 L round-bottomed flask, methyl 1-(4′-(trifluoromethylsulfonyloxy)biphenyl-4-yl)cyclopropanecarboxylate (2.8 g, 6.99 mmol), 4,4,4′,4′,5,5,5′,5′-octamethyl-2,2′-bi(1,3,2-dioxaborolane) (2.13 g, 8.39 mmol, 1.2 eq) and potassium acetate (2.06 g, 21.0 mmol, 3.0 eq) were combined with dioxane (10 mL) to give a brown suspension. PdCl₂(dppf) (457 mg, 559 μmol, 0.08 eq) was added and the reaction mixture was heated to 90° C. and stirred for 4 h followed by stirring at 25° C. for 12 h. The reaction was diluted with EtOAc, filtered through Celite and stripped in vacuo. The crude material was purified by filtering over silica gel under vacuum eluting with Hex/EtOAc 1:1. The filtrate was stripped to an off-white powder. The material was re-purified by flash chromatography (silica gel, 300 g, 0% to 20% EtOAc in heptane) to afford 2.11 g (78%) of 1-[4′-(4,4,5,5-tetramethyl-[1,3,2]dioxaborolan-2-yl)-biphenyl-4-yl]-cyclopropanecarboxylic acid methyl ester as an off white crystalline solid. (M+H)⁺=379.0 (m/e). ¹H NMR (DMSO-d₆) 6 ppm 7.72-7.94 (m, 2H), 7.53-7.72 (m, 4H), 7.26-7.53 (m, J=8.6 Hz, 2H), 3.65 (s, 3H), 1.40-1.64 (m, 2H), 1.27-1.40 (m, 12H), 1.21-1.27 (m, 2H).

To a 50 mL tube was added methyl 1-(4′-(4,4,5,5-tetramethyl-1,3,2-dioxaborolan-2-yl)biphenyl-4-yl)cyclopropanecarboxylate (2.033 g, 5.37 mmol), 4-bromo-1-methyl-1H-pyrazol-5-amine (1.23 g, 6.99 mmol, 1.3 eq), X-Phos (769 mg, 1.61 mmol, 0.30 eq) and K₃PO₄ (3.42 g, 16.1 mmol, 3.0 eq) in toluene (31 mL) and water (6 mL). Pd(OAc)₂ (181 mg, 806 μmol, 0.15 eq) was added and the tube was sealed. The reaction was purged with argon and heated at 100° C. for 21 h. The reaction was cooled and diluted with EtOAc and water. The suspension was filtered and the phases separated. The organic layers were dried over Na₂SO₄ and concentrated in vacuo. The crude material was purified by flash chromatography (silica gel, 300 g, 0% to 5% methanol in dichloromethane). The product was isolated as an impure oily red solid. The material was taken up in a minimal amount of warm dichloromethane and precipitated with hexanes. The suspension was filtered and the filtrate stripped in vacuo. The residue was taken up in a minimal amount of warm dichloromethane and precipitated with hexanes. The supernatant was decanted from the tan solid and the solid was washed with hexanes. The combined solids were dried under vacuum to afford 738 mg (39%) of 1-[4′-(5-amino-1-methyl-1H-pyrazol-4-yl)-biphenyl-4-yl]-cyclopropanecarboxylic acid methyl ester. (M+H)⁺=348.2 (m/e); ¹H NMR (DMSO-d₆) 6 ppm 7.37-7.63 (m, 8H), 7.11 (s, 1H), 5.40 (s, 2H), 3.52-3.61 (m, 6H) 1.49 (d, J=3.0 Hz, 2H), 1.22 (d, J=3.0 Hz, 2H).

In a 25 mL tube, methyl 1-(4′-(5-amino-1-methyl-1H-pyrazol-4-yl)-biphenyl-4-yl)-cyclopropanecarboxylate (94 mg, 271 μmol) was combined with 2 mL of dichloromethane and 5 mL of toluene. To this suspension was added triphosgene (120 mg, 406 μmol, 1.5 eq) followed by TEA (151 μL, 1.08 mmol, 4 eq). The tube was sealed and the mixture was stirred at 90° C. for 30 mins. The mixture was cooled and (R)-butan-2-ol (40.1 mg, 49.8 μL, 541 μmol, 2 eq) was added. The reaction mixture was sealed and heated to 90° C. for 2 h. The reaction was partitioned between EtOAc and saturated NH₄Cl. The organic layer was washed with H₂O (25 mL), sat NaCl (25 mL), dried over Na₂SO₄ and concentrated in vacuo. The crude material was purified by flash chromatography (silica gel, 40 g, 100% EtOAc) to afford 60 mg (50%) of 1-{4′-[5-((R))-sec-butoxycarbonylamino)-1-methyl-1H-pyrazol-4-yl]-biphenyl-4-yl}-cyclopropanecarboxylic acid methyl ester as an off white solid. (M+H)⁺=448.0 (m/e).

In a 250 mL round-bottomed flask, (R)-methyl 1-(4′-(5-(sec-butoxycarbonylamino)-1-methyl-1H-pyrazol-4-yl)biphenyl-4-yl)cyclopropanecarboxylate (60 mg, 134 μmol) was combined with tetrahydrofuran (3 mL) and methanol (3.00 mL) to give a light yellow solution. 1M NaOH (1.34 mLl, 1.34 mmol, 10 eq) was added and the reaction mixture was stirred at 45° C. for 4 h. The reaction was concentrated and acidified with 1M HCl. The residue was partitioned between dichloromethane and water. The aqueous layer was back-extracted with dichloromethane (10 mL). The organic layers were combined, washed with H₂O (10 mL) and dried in vacuo to afford 54 mg (93%) of (R)-1-{4′-[5-(sec-butoxycarbonylamino)-1-methyl-1H-pyrazol-4-yl]-biphenyl-4-yl}-cyclopropanecarboxylic acid as a white powder. LC/MS calcd for C₂₅H₂₇N₃O₄ (m/e) 433.0, obsd 434.1 (M+H, ES+); ¹H NMR (DMSO-d₆) δ ppm 12.32 (br. s., 1H), 9.38 (br. s., 1H), 7.80 (s, 1H), 7.50-7.69 (m, 6H), 7.38 (d, J=8.3 Hz, 2H), 4.69 (br. s., 1H), 3.65 (s, 3H), 1.58 (br. s., 2H), 1.33-1.52 (m, J=2.6 Hz, 2H), 1.22 (br. s., 3H), 1.14 (m, J=2.6 Hz, 2H), 0.91 (br. s., 3H).

### Example 15

### (R)-1-{4′-[5-(1,2-Dimethyl-propoxycarbonylamino)-1-methyl-1H-pyrazol-4-yl]-biphenyl-4-yl}-cyclopropanecarboxylic acid

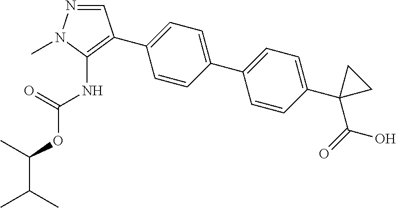 [SMILES: CC(C)[C@@H](C)OC(=O)Nc1c(-c2ccc(-c3ccc(C4(C(=O)O)CC4)cc3)cc2)cnn1C]

Preparation by a similar procedure to Example 14, except substituting (R)-3-methylbutan-2-ol for (R)-butan-2-ol, afforded (R)-1-{4′-[5-(1,2-dimethyl-propoxycarbonylamino)-1-methyl-1H-pyrazol-4-yl]-biphenyl-4-yl}-cyclopropanecarboxylic acid. LC/MS calcd for C₂₆H₂₉N₃O₄ (m/e) 447.0, obsd 448.2 (M+H, ES+); ¹H NMR (DMSO-d₆) δ ppm 12.34 (br. s., 1H), 9.37 (br. s., 1H), 7.81 (s, 1H), 7.49-7.68 (m, 6H), 7.38 (d, J=8.3 Hz, 2H), 4.58 (br. s., 1H), 3.65 (s, 3H), 1.82 (br. s., 1H), 1.44 (br. s., 2H), 1.03-1.30 (m, 5H), 0.92 (br. s., 6H).

### Example 16

### (R)-1-{4′-[5-((1-(2-Fluorophenyl)ethoxy)carbonylamino)-1-methyl-1H-pyrazol-4-yl]-biphenyl-4-yl}-cyclopropanecarboxylic acid

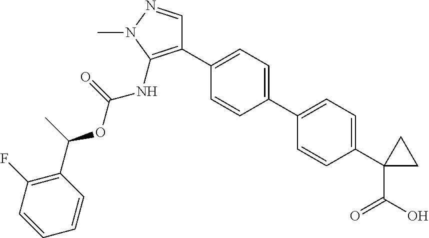 [SMILES: C[C@@H](OC(=O)Nc1c(-c2ccc(-c3ccc(C4(C(=O)O)CC4)cc3)cc2)cnn1C)c1ccccc1F]

Preparation by a similar procedure to Example 14, except substituting (R)-1-(2-fluorophenyl) ethanol for (R)-butan-2-ol, afforded (R)-1-{4′-[5-((1-(2-fluorophenyl)ethoxy)carbonylamino)-1-methyl-1H-pyrazol-4-yl]-biphenyl-4-yl}-cyclopropanecarboxylic acid. LC/MS calcd for C₂₉H₂₆FN₃O₄ (m/e) 499.0, obsd 500.1 (M+H, ES+); ¹H NMR (DMSO-d₆) δ ppm 12.37 (br. s., 1H), 9.67 (s, 1H), 7.80 (s, 2H), 7.45-7.70 (m, 8H), 7.39 (d, 2H), 7.19-7.33 (m, 1H), 5.97 (d, J=5.7 Hz, 1H), 3.62 (s, 3H), 1.57 (m, 2H), 1.38-1.52 (m, 2H), 1.22 (s, 1H), 1.15 (d, 2H).

### Example 17

### (R)-1-{4′-[1-Methyl-5-((1-(3-(trifluoromethyl)phenyl)ethoxy)carbonylamino)-1H-pyrazol-4-yl]-biphenyl-4-yl}-cyclopropanecarboxylic acid

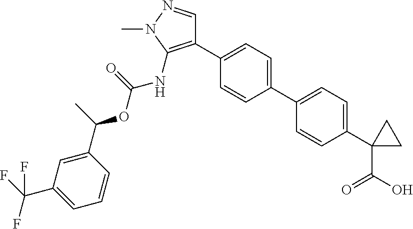 [SMILES: C[C@@H](OC(=O)Nc1c(-c2ccc(-c3ccc(C4(C(=O)O)CC4)cc3)cc2)cnn1C)c1cccc(C(F)(F)F)c1]

Preparation by a similar procedure to Example 14, except substituting (R)-1-(3-(trifluoromethyl)-phenyl)ethanol for (R)-butan-2-ol, afforded (R)-1-{4′-[1-methyl-5-((1-(3-(trifluoromethyl)-phenyl)ethoxy)-carbonylamino)-1H-pyrazol-4-yl]-biphenyl-4-yl}-cyclopropanecarboxylic acid. LC/MS calcd for C₃₀H₂₆F₃N₃O₄ (m/e) 549.0, obsd 550.1 (M+H, ES+); ¹H NMR (DMSO-d₆) δ ppm 12.34 (br. s., 1H), 9.71 (br. s., 1H), 7.45-7.85 (m, 10H), 7.39 (d, J=8.3 Hz, 3H), 5.87 (d, J=6.0 Hz, 1H), 3.65 (s, 3H), 1.57 (d, J=6.0 Hz, 2H), 1.39-1.51 (m, 2H), 1.22 (br. s., 1H), 1.09-1.19 (m, 2H).

### Example 18

### 1-{4′-[5-((1-(4-fluorophenyl)ethoxy)carbonylamino)-1-methyl-1H-pyrazol-4-yl]-biphenyl-4-yl}-cyclopropanecarboxylic acid

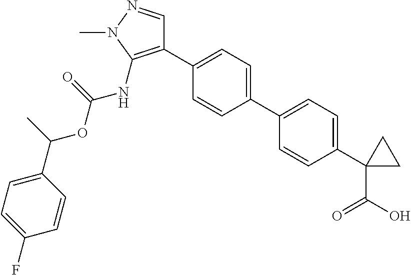 [SMILES: CC(OC(=O)Nc1c(-c2ccc(-c3ccc(C4(C(=O)O)CC4)cc3)cc2)cnn1C)c1ccc(F)cc1]

In a 250 ml, round-bottomed flask, methyl 1-(4′-(5-((1-(4-fluorophenyl)ethoxy) carbonylamino)-1-methyl-1H-pyrazol-4-yl)biphenyl-4-yl)cyclopropanecarboxylate (66 mg, 129 μmol) was combined with THF (2 mL) and methanol (2 mL) to give a yellow solution. NaOH (1.29 mL, 1.29 mmol, 10 eq) was added and the reaction mixture was heated at 45° C. and stirred for 3.5 h. The reaction was concentrated and acidified with 1M HCl. The reaction was partitioned between dichloromethane and water. The aqueous layer was back-extracted with dichloromethane (10 mL). The organic layers were combined, washed with H₂O (10 mL), dried over Na₂SO₄ and concentrated in vacuo to a white powder. The crude material was purified by flash column chromatography (silica gel, 12 g, 0% to 5% methanol in dichloromethane) to afford 29 mg (46%) of 1-(4′-(5-((1-(4-fluorophenyl)ethoxy)carbonylamino)-1-methyl-1H-pyrazol-4-yl)biphenyl-4-yl)-cyclopropanecarboxylic acid as an off white powder. LC/MS calcd for C₂₉H₂₆FN₃O₄ (m/e) 499.0, obsd 498.0 (M−H, ES−); ¹H NMR (DMSO-d₆) δ ppm 12.36 (br. s., 1H), 9.62 (br. s., 1H), 7.82 (s, 1H), 7.60 (d, J=8.1 Hz, 4H), 7.51 (d, J=7.8 Hz, 3H), 7.35-7.44 (m, 2H), 7.26 (t, J=7.8 Hz, 2H), 6.94-7.18 (m, 1H), 5.79 (d, J=5.8 Hz, 1H), 3.68 (br. s., 3H), 1.56 (d, J=5.8 Hz, 2H), 1.42-1.51 (m, 2H), 1.21-1.33 (br. m, 1H), 1.11-1.21 (m, 2H).

### Example 19

### Preparation of 1-{4′-[5-(cyclobutoxycarbonylamino)-1-methyl-1H-pyrazol-4-yl]-biphenyl-4-yl}-cyclopropanecarboxylic acid

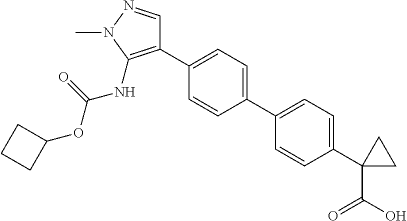 [SMILES: Cn1ncc(-c2ccc(-c3ccc(C4(C(=O)O)CC4)cc3)cc2)c1NC(=O)OC1CCC1]

Preparation by a similar procedure to Example 18, except substituting cyclobutanol for 1-(4-fluorophenyl)ethanol, afforded 1-{4′-[5-(cyclobutoxycarbonylamino)-1-methyl-1H-pyrazol-4-yl]-biphenyl-4-yl}-cyclopropanecarboxylic acid. LC/MS calcd for C₂₅H₂₅N₃O₄ (m/e) 431.0, obsd 432.0 (M+H, ES+); ¹H NMR (Methanol-d₄) δ ppm 7.77 (s, 1H), 7.52-7.72 (m, 5H), 7.44 (d, J=8.3 Hz, 2H), 5.01 (br. s., 1H), 3.79 (s, 3H), 2.40 (br.s., 2H), 2.05-2.29 (m, 2H), 1.78-1.94 (m, 1H), 1.46-1.76 (m, 3H), 1.12-1.42 (m, 2H).

### Example 20

### 1-{4′-[1-Methyl-5-((oxetan-3-yloxy)carbonylamino)-1H-pyrazol-4-yl]-biphenyl-4-yl}-cyclopropanecarboxylic acid

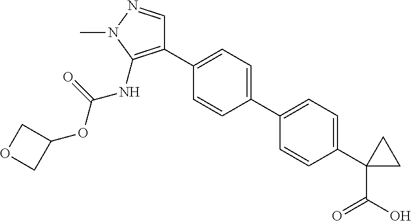 [SMILES: Cn1ncc(-c2ccc(-c3ccc(C4(C(=O)O)CC4)cc3)cc2)c1NC(=O)OC1COC1]

Preparation by a similar procedure to Example 18, except substituting oxetan-3-ol for 1-(4-fluorophenyl)ethanol, afforded 1-{4′-[1-methyl-5-((oxetan-3-yloxy)carbonylamino)-1H-pyrazol-4-yl]-biphenyl-4-yl}-cyclopropanecarboxylic acid. LC/MS calcd for C₂₄H₂₃N₃O₅ (m/e) 433.0, obsd 434.1 (M+H, ES+); ¹H NMR (DMSO-d₆) δ ppm 12.33 (br. s., 1H), 7.93 (s, 1H), 7.66-7.73 (m, J=8.3 Hz, 2H), 7.61 (d, J=8.3 Hz, 2H), 7.53-7.59 (m, J=8.1 Hz, 2H), 7.42 (d, J=8.1 Hz, 2H), 5.41 (br. s., 1H), 4.85-4.94 (m, 1H), 3.90 (t, 1H), 3.66-3.83 (m, 6H), 3.57 (br. d., 1H), 1.42-1.62 (m, 2H), 1.13-1.22 (m, 2H).

### Example 21

### [4-(2-Fluoro-phenyl)-2-methyl-2H-pyrazol-3-yl]-carbamic acid (R)-1-(2-chloro-phenyl)-ethyl ester

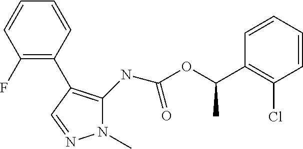 [SMILES: C[C@@H](OC(=O)Nc1c(-c2ccccc2F)cnn1C)c1ccccc1Cl]

This compound was prepared by the chiral SFC separation of the corresponding racemate in Example 13. The separation conditions are the following: chiral WHELKO column, 10% to 65% methanol in CO₂. The second fraction was concentrated to give the desired compound. LC/MS calcd for C₁₉H₁₇C1FN₃O₂ (m/e) 373.0, obsd 374.0 (M+H, ES+).

### Example 22

### [4-(2-Fluoro-phenyl)-2-methyl-2H-pyrazol-3-yl]-carbamic acid (S)-1-(2-chloro-phenyl)-ethyl ester

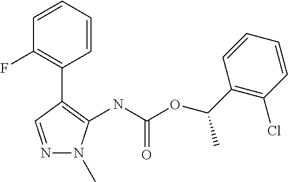 [SMILES: C[C@H](OC(=O)Nc1c(-c2ccccc2F)cnn1C)c1ccccc1Cl]

This compound was prepared by the chiral SFC separation of the corresponding racemate in Example 13. The separation conditions are the following: chiral WHELKO column, 10% to 65% methanol in CO₂. The first fraction was concentrated to give the desired compound. LC/MS calcd for C₁₉H₁₇C1FN₃O₂ (m/e) 373.0, obsd 374.0 (M+H, ES+).

### Example 23

### [4-(2-Fluoro-phenyl)-2-methyl-2H-pyrazol-3-yl]-carbamic acid (R)-1-(3-trifluoromethyl-phenyl)-ethyl ester

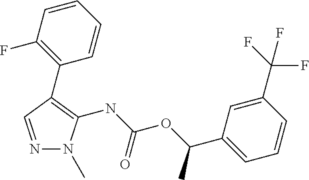 [SMILES: C[C@@H](OC(=O)Nc1c(-c2ccccc2F)cnn1C)c1cccc(C(F)(F)F)c1]

This compound was prepared by the chiral SFC separation of the corresponding racemate in Example 12. The separation conditions are the following: chiral WHELKO column, 10% to 65% methanol in CO₂. The second fraction was concentrated to give the desired compound. LC/MS calcd for C₂₀H₁₇F₄N₃O₂ (m/e) 407.0, obsd 408.0 (M+H, ES+).

### Example 24

### 1-{4′-[5-(3-Benzyl-ureido)-1-methyl-1H-pyrazol-4-yl]-biphenyl-4-yl}-cyclopropanecarboxylic acid

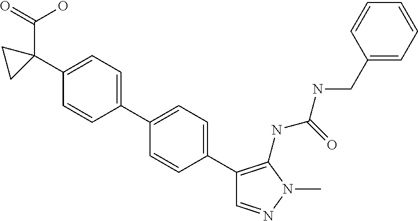 [SMILES: Cn1ncc(-c2ccc(-c3ccc(C4(C(=O)O)CC4)cc3)cc2)c1NC(=O)NCc1ccccc1]

Sodium hydride (40.2 mg, 1.01 mmol) in mineral oil was added to a solution of 1-(4′-(5-amino-1-methyl-1H-pyrazol-4-yl)biphenyl-4-yl)cyclopropanecarboxylic acid methyl ester (233 mg, 0.67 mmol) in DMF (8 mL) at room temperature under nitrogen atmosphere and the mixture was stirred for 5 minutes. The reaction mixture turned to a red brown solution after addition of sodium hydride. Then, benzyl isocyanate (89.2 mg, 0.67 mmol) was added and the reaction mixture was heated to 80° C. and stirred for 3 h at which time LC/MS analysis indicated the presence of the desired product. The mixture was cooled to room temperature and diluted with water and the organic compound was extracted into ethyl acetate (2×50 mL). The combined extracts were washed with water and brine solution and dried over anhydrous MgSO₄. Filtration and concentration gave the crude residue which was purified using an ISCO (40 g) column chromatography eluting with ethyl acetate in hexanes (0-100%). The desired fractions were combined and the solvent was removed under vacuum to obtain 1-(4′-(5-(3-benzylureido)-1-methyl-1H-pyrazol-4-yl)biphenyl-4-yl)cyclopropanecarboxylic acid methyl ester (105 mg, 32% yield) as a brown oil. LC/MS calcd. for C₂₉H₂₈N₄O₃ (m/e) 480, obsd. 481.1 (M+H, ES+).

To a solution of 1-(4′-(5-(3-benzylureido)-1-methyl-1H-pyrazol-4-yl)biphenyl-4-yl)cyclopropanecarboxylic acid methyl ester (105 mg, 0.22 mmol) in THF (6 mL) and ethanol (6 mL) was added an excess of 1.0 M solution of sodium hydroxide (5.83 mL, 5.83 mmol) at room temperature. The resulting light yellow solution was stirred for 15 h at room temperature at which time LC/MS analysis indicated the absence of starting material. Then, the solvent was removed and the basic aqueous layer was diluted with water and the neutral impurities were extracted into ethyl acetate. The basic solution was neutralized with 1.0 N HCl. The resulting solids were collected by filtration and washed with water and hexanes. After air drying, 1-(4′-(5-(3-benzylureido)-1-methyl-1H-pyrazol-4-yl)biphenyl-4-yl)cyclopropanecarboxylic acid (59 mg, 57% yield) was isolated as an off-white solid. ¹H NMR (DMSO-d₆) δ 12.34 (br. s., 1H), 8.27 (s, 1H), 7.81 (s, 1H), 7.36-7.73 (m, 9H), 7.19-7.36 (m, 4H), 7.03 (br. s., 1H), 4.31 (d, J=5.8 Hz, 2H), 3.60-3.74 (m, 3H), 1.39-1.58 (m, 2H), 1.04-1.30 (m, 2H). LC/MS calcd. for C₂₈H₂₆N₄O₃ (m/e) 466, obsd. 467.3 (M+H, ES+).

### Example 25

### 1-{4′-[1-Methyl-5-((S)-3-phenyl-butyrylamino)-1H-pyrazol-4-yl]-biphenyl-4-yl}-cyclopropanecarboxylic acid

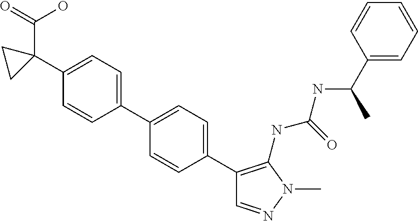 [SMILES: C[C@@H](NC(=O)Nc1c(-c2ccc(-c3ccc(C4(C(=O)O)CC4)cc3)cc2)cnn1C)c1ccccc1]

To a solution of (S)-3-phenylbutanoic acid (181 mg, 1.1 mmol) in toluene (5 mL) was added an excess of thionyl chloride (2.62 g, 1.61 mL, 22.0 mmol) at room temperature. The resulting colorless solution was stirred for 15 h at reflux temperature. Then, the reaction mixture was cooled to room temperature and the solvent was removed under vacuum. The resulting residue was azeotrophed one time with toluene and dried under high vacuum.

To a suspension of 1-(4′-(5-amino-1-methyl-1H-pyrazol-4-yl)biphenyl-4-yl)cyclopropanecarboxylic acid methyl ester (174 mg, 0.5 mmol) and DMAP (67.2 mg, 0.55 mmol) in dichloromethane (5 mL) was added a solution of (S)-3-phenylbutanoyl chloride (above prepared) (95.9 mg, 0.525 mmol) in dichloromethane (5 mL). The resulting light brown suspension was stirred for 2 days at room temperature under nitrogen atmosphere at which time TLC analysis indicated the absence of starting material. The mixture was diluted with water and dichloromethane and the two layers were separated and the aqueous layer was extracted one more time with dichloromethane. The combined extracts were washed with brine solution and dried over anhydrous MgSO₄. Filtration and concentration gave the crude residue which was purified using an ISCO (80 g) column chromatography eluting with ethyl acetate in hexanes (0-100%). The desired fractions were combined and the solvent was removed under vacuum to obtain (S)-1-(4′-(1-methyl-5-(3-phenylbutanamido)-1H-pyrazol-4-yl)biphenyl-4-yl)cyclopropanecarboxylic acid methyl ester (50 mg, 20% yield) as a white solid. LC/MS calcd. for C₃₁H₃₁N₃O₃ (m/e) 493, obsd. 494.1 (M+H, ES+).

To a solution of (S)-1-(4′-(1-methyl-5-(3-phenylbutanamido)-1H-pyrazol-4-yl)biphenyl-4-yl)-cyclopropanecarboxylic acid methyl ester (43 mg, 0.087 mmol) in THF (5 mL) and EtOH (5 mL) was added an excess of a 1 M solution of sodium hydroxide (1.74 mL, 1.74 mmol) at room temperature. The resulting solution was stirred for 15 h at room temperature at which time TLC and LCMS analysis indicated the absence of starting material. Then, the solvent was removed under vacuum and the aqueous layer was diluted with water and neutralized with 1 N HCl. The resulting solids were collected by filtration and washed with water and hexanes. After air drying, (S)-1-(4′-(1-methyl-5-(3-phenylbutanamido)-1H-pyrazol-4-yl)biphenyl-4-yl)cyclopropanecarboxylic acid (32 mg, 76.6% yield) was isolated as an off-white solid. ¹H NMR (DMSO-d₆) δ 12.35 (br. s., 1H), 9.95 (s, 1H), 7.79 (s, 1H), 7.53-7.71 (m, 4H), 7.47 (d, J=8.3 Hz, 2H), 7.42 (d, J=8.3 Hz, 2H), 7.30-7.38 (m, 4H), 7.25 (td, J=6.0, 2.6 Hz, 1H), 3.27-3.43 (m, 4H), 2.63-2.86 (m, 2H), 1.41-1.58 (m, 2H), 1.30 (d, J=7.0 Hz, 3H), 1.13-1.23 (m, 2H). LC/MS calcd for C₃₀H₂₉N₃O₃ (m/e) 479, obsd. 480.3 (M+H, ES+).

### Example 26

### Calcium Flux Assay Using Fluorometric Imaging Plate Reader (FLIPR)

Cell Culture Conditions: The ChemiScreen Calcium-optimized stable cell line containing the human recombinant LPA1 Lysophospholipid receptor was purchased from Chemicon International, Inc./Millipore. The cells were cultured in DMEM-high glucose supplemented with 10% fetal bovine serum, 2 mM glutamine, 100 U/mL penicillin/100 μg/mL streptomycin, 1× non-essential amino acids, 10 mM HEPES and 0.25 mg/mL Geneticin. Cells were harvested with trypsin-EDTA and counted using ViaCount reagent. The cell suspension volume was adjusted to 2.0×10⁵ cells/mL with complete growth media. Aliquots of 50 μL were dispensed into 384 well black/clear tissue culture treated plates (BD) and the microplates were placed in a 37° C. incubator overnight. The following day plates were used in the assay.

Dye Loading and Assay: Loading Buffer (FLIPR Calcium-4, Molecular Devices) was prepared by dissolving the contents of one bottle into 100 mL Hank's Balanced Salt Solution containing 20 mM HEPES and 2.5 mM probenecid. Plates were loaded onto Biotek plate washer and growth media was removed and replaced with 20 L of Hank's Balanced Salt Solution containing 20 mM HEPES and 2.5 mM probenecid, followed by 25 μL of Loading Buffer. The plates were then incubated for 30 minutes at 37° C.

During the incubation, test compounds were prepared by adding 90 μL of HBSS/20 mM HEPES/0.1% BSA buffer to 2 μL of serially diluted compounds. To prepare serial dilutions, 10 mM stocks of compounds were prepared in 100% DMSO. The compound dilution plate was set up as follows: well #1 received 29 μL of stock compound and 31 μL DMSO; wells 2-10 received 40 μL of DMSO; mixed and transferred 20 μL of solution from well #1 into well #2; continued with 1:3 serial dilutions out 10 steps; transferred 2 μL of diluted compound into duplicate wells of 384 well “assay plate” and then added the 90 μL of buffer.

After incubation, both the cell and “assay” plates were brought to the FLIPR and 20 μL of the diluted compounds were transferred to the cell plates by the FLIPR. Compound addition was monitored by FLIPR to detect any agonist activity of the compounds. Plates were then incubated for 30 minutes at room temperature protected from light. After the incubation, plates were returned to the FLIPR and 20 μL of 4.5× concentrated agonist was added to the cell plates. During the assay, fluorescence readings were taken simultaneously from all 384 wells of the cell plate every 1.5 seconds. Five readings were taken to establish a stable baseline, then 20 μL of sample was rapidly (30 μL/sec) and simultaneously added to each well of the cell plate. The fluorescence was continuously monitored before, during and after sample addition for a total elapsed time of 100 seconds. Responses (increase in peak fluorescence) in each well following agonist addition was determined. The initial fluorescence reading from each well, prior to ligand stimulation, was used as zero baseline value for the data from that well. The responses were expressed as % inhibition of the buffer control. The IC₅₀ value, defined as the concentration of a compound required for 50% inhibition of the buffer control, was calculated by fitting the percent inhibition data for 10 concentrations to a sigmoidal dose-response (4 parameter logistic) model using Genedata Condoseo program [model 205, F(x)=(A+(B−A)/(1+((C/x)̂D))))] and the results shown in Table 1 below:

TABLE 1
LPA1R and LPA3R antagonist activities

| | LPA1R IC₅₀ (μM) | LPA3R |
|---|---|---|
| Example # | or (inhibition % @ μM) | IC₅₀ (μM) |
| 1 | 17.50 (64.8% @ 30) | >30 |
| 2 | 0.035 | >30 |
| 3 | 3.59 | >30 |
| 4 | 1.59 (65.9% @ 30) | >30 |
| 5 | 0.02 | 8.35 |
| 6 | 0.112 | 2.47 |
| 7 | >30 | >30 |
| 8 | >30 | >30 |
| 9 | >30 | >30 |
| 10 | >30 | >30 |
| 11 | 3.93 | >30 |
| 12 | 24.65 (50.5% @ 30) | >30 |
| 13 | 1.4 | >30 |
| 14 | 0.038 | >30 |
| 15 | 0.107 | >30 |
| 16 | 0.024 | 5.8 |
| 17 | 0.042 | 0.478 |
| 18 | 0.057 | 8.26 |
| 19 | 0.176 | >30 |
| 20 | >30 | >30 |
| 21 | 0.644 (97.2% @ 30) | >30 |
| 22 | >30 | >30 |
| 23 | >30 | >30 |
| 24 | 0.58 (82.8% @ 10) | >30 |
| 25 | 0.88 (85.5% @ 30) | >30 |

It is to be understood that the invention is not limited to the particular embodiments of the invention described above, as variations of the particular embodiments may be made and still fall within the scope of the appended claims.

1. A compound of formula (I): 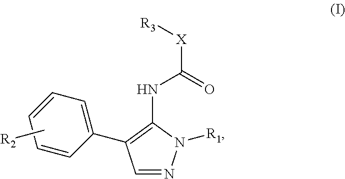
wherein:
X is oxygen, nitrogen or carbon
R1 is lower alkyl;
R₂ is hydrogen, halogen, —CH₂C(O)OH, alkoxy, cycloalkylcarboxylic acid, unsubstituted phenyl or phenyl substituted with halogen, —CH₂C(O)OH, cyclopropanecarboxylic acid, cyclopropanecarboxylic acid ethyl ester, methanesulfonylaminocarbonyl or tetrazole; and
R₃ is cyclobutyl, oxetanyl, unsubstituted lower alkyl, lower alkyl substituted with unsubstituted phenyl or lower alkyl substituted with phenyl substituted with halogen or —CF₃,
or a pharmaceutically acceptable salt thereof.

2. The compound according to claim 1, wherein X is oxygen.

3. The compound according to claim 1, wherein R₁ is methyl.

4. The compound according to claim 1, wherein R₂ is hydrogen, F, Cl, —CH₂C(O)OH, methoxy, ethoxy, cyclopropanecarboxylic acid, cyclohexaneacetic acid or unsubstituted phenyl.

5. The compound according to claim 1, wherein R₂ is phenyl substituted with —CH₂C(O)OH, cyclopropanecarboxylic acid or cyclopropanecarboxylic acid ethyl.

6. The compound according to claim 1, wherein R₃ is cyclobutyl, oxetanyl or unsubstituted lower alkyl.

7. The compound according to claim 1, wherein R₃ is lower alkyl substituted with phenyl substituted with F, Cl or —CF₃.

8. The compound according to claim 1, wherein said compound is:
2-Methyl-4-phenyl-2H-pyrazol-3-yl-carbamic acid (R)-1-phenyl-ethyl ester;
{4′-[1-Methyl-5-((R)-1-phenyl-ethoxycarbonylamino)-1H-pyrazol-4-yl]-biphenyl-4-yl}-acetic acid;
(2-Methyl-4-phenyl-2H-pyrazol-3-yl)-carbamic acid 1-(2-chloro-phenyl)-ethyl ester;
1-{4′-[1-Methyl-5-((R)-1-phenyl-ethoxycarbonylamino)-1H-pyrazol-4-yl]-biphenyl-4-yl}-cyclopropanecarboxylic acid ethyl ester;
1-{4′-[1-Methyl-5-((R)-1-phenyl-ethoxycarbonylamino)-1H-pyrazol-4-yl]-biphenyl-4-yl}-cyclopropanecarboxylic acid;
1-(4′-{5-[1-(2-Chloro-phenyl)-ethoxycarbonylamino]-1-methyl-1H-pyrazol-4-yl}-biphenyl-4-yl)-cyclopropanecarboxylic acid;
(4-Biphenyl-4-yl-2-methyl-2H-pyrazol-3-yl)-carbamic acid (R)-1-phenyl-ethyl ester;
[4-(4-Methoxy-phenyl)-2-methyl-2H-pyrazol-3-yl]-carbamic acid (R)-1-phenyl-ethyl ester;
1-{4-[1-Methyl-5-((R)-1-phenyl-ethoxycarbonylamino)-1H-pyrazol-4-yl]-phenyl}-cyclopropanecarboxylic acid;
{4-[1-Methyl-5-((R)-1-phenyl-ethoxycarbonylamino)-1H-pyrazol-4-yl]-phenyl}-acetic acid;
[4-(4-Fluoro-phenyl)-2-methyl-2H-pyrazol-3-yl]-carbamic acid 1-(2-chloro-phenyl)-ethyl ester;
[4-(2-Fluoro-phenyl)-2-methyl-2H-pyrazol-3-yl]-carbamic acid 1-(3-trifluoromethyl-phenyl)-ethyl ester;
[4-(2-Fluoro-phenyl)-2-methyl-2H-pyrazol-3-yl]-carbamic acid 1-(2-chloro-phenyl)-ethyl ester;
(R)-1-{4′-[5-(sec-Butoxycarbonylamino)-1-methyl-1H-pyrazol-4-yl]-biphenyl-4-yl}-cyclopropanecarboxylic acid;
(R)-1-{4′-[5-(1,2-Dimethyl-propoxycarbonylamino)-1-methyl-1H-pyrazol-4-yl]-biphenyl-4-yl}-cyclopropanecarboxylic acid;
(R)-1-{4′-[5-((1-(2-Fluorophenyl)ethoxy)carbonylamino)-1-methyl-1H-pyrazol-4-yl]-biphenyl-4-yl}-cyclopropanecarboxylic acid;
(R)-1-{4′-[1-Methyl-5-((1-(3-(trifluoromethyl)phenyl)ethoxy)carbonylamino)-1H-pyrazol-4-yl]-biphenyl-4-yl}-cyclopropanecarboxylic acid;
1-{4′-[5-((1-(4-fluorophenyl)ethoxy)carbonylamino)-1-methyl-1H-pyrazol-4-yl]-biphenyl-4-yl}-cyclopropanecarboxylic acid;
1-{4′-[5-(cyclobutoxycarbonylamino)-1-methyl-1H-pyrazol-4-yl]-biphenyl-4-yl}-cyclopropanecarboxylic acid;
1-{4′[1-Methyl-5-((oxetan-3-yloxy)carbonylamino)-1H-pyrazol-4-yl]-biphenyl-4-yl}-cyclopropanecarboxylic acid;
[4-(2-Fluoro-phenyl)-2-methyl-2H-pyrazol-3-yl]-carbamic acid (R)-1-(2-chloro-phenyl)-ethyl ester;
[4-(2-Fluoro-phenyl)-2-methyl-2H-pyrazol-3-yl]-carbamic acid (S)-1-(2-chloro-phenyl)-ethyl ester;
[4-(2-Fluoro-phenyl)-2-methyl-2H-pyrazol-3-yl]-carbamic acid (R)-1-(3-trifluoromethyl-phenyl)-ethyl ester;
1-{4′-[5-(3-Benzyl-ureido)-1-methyl-1H-pyrazol-4-yl]-biphenyl-4-yl}-cyclopropanecarboxylic acid; or
1-{4′-[1-Methyl-5-((S)-3-phenyl-butyrylamino)-1H-pyrazol-4-yl]-biphenyl-4-yl}-cyclopropanecarboxylic acid.

9. (canceled)

10. A pharmaceutical composition, comprising a therapeutically effective amount of a compound according to claim 1 and a therapeutically inert carrier.

11-13. (canceled)

14. A method for the treatment or prophylaxis of pulmonary fibrosis, comprising the step of administering an effective amount of a compound according to claim 1 to a patient in need thereof.

15. (canceled)
